(19) United States
(12) Patent Application Publication
(10) Pub. No.: US 2011/0003330 A1
(43) Pub. Date: Jan. 6, 2011

(54) MICROFLUIDIC DEVICE

(76) Inventor: Gary P. Durack, Urbana, IL (US)

Correspondence Address: Woodard, Emhardt, Moriarty, McNett & Henry LLP;Sony Corporation, 111 Monument Circle, Suite 3700, Indianapolis, IN, 46204-5137, US

(21) Appl. No.: 12/831,107

(22) Filed: Jul. 6, 2010

Related U.S. Application Data
(60) Provisional application No. 61/223,415, filed on Jul. 7, 2009.
(60) Provisional application No. 61/223,411, filed on Jul. 7, 2009.
(60) Provisional application No. 61/223,742, filed on Jul. 8, 2009.
(60) Provisional application No. 61/223,081, filed on Jul. 6, 2009.
(60) Provisional application No. 61/223,084, filed on Jul. 6, 2009.
(60) Provisional application No. 61/223,086, filed on Jul. 6, 2009.
(60) Provisional application No. 61/223,088, filed on Jul. 6, 2009.
(60) Provisional application No. 61/223,085, filed on Jul. 6, 2009.
(60) Provisional application No. 61/223,405, filed on Jul. 7, 2009.
(60) Provisional application No. 61/223,423, filed on Jul. 7, 2009.
(60) Provisional application No. 61/223,425, filed on Jul. 7, 2009.

(51) Int. Cl. C12M 1/34; C12Q 1/04

(52) U.S. Cl. 435/34; 435/287.1

(57) ABSTRACT

The present disclosure relates to microfluidic devices adapted for facilitating cytometry analysis of particles flowing therethrough. In certain embodiments, the microfluidic devices have onboard data storage capabilities. In certain other embodiments, the microfluidic devices have onboard anticoagulants. In certain other embodiments, the microfluidic devices have onboard test and control channels. In certain other embodiments, the microfluidic devices have integrated collection media. In certain other embodiments, the microfluidic devices have multiple onboard test channels. In certain other embodiments, the microfluidic devices have localized temperature control. In certain other embodiments, the microfluidic devices have anatomy simulating regions. In certain other embodiments, the microfluidic devices have complete assay capabilities. In certain other embodiments, the microfluidic devices have dissociable sections. In certain other embodiments, the microfluidic devices have means for performing functional assays.

### CROSS-REFERENCE TO RELATED APPLICATIONS

The present application claims the benefit of the following: U.S. Provisional Patent Application No. 61/223,415, which was filed Jul. 7, 2009, U.S. Provisional Patent Application No. 61/223,411, which was filed Jul. 7, 2009, U.S. Provisional Patent Application No. 61/223,742, which was filed Jul. 8, 2009, U.S. Provisional Patent Application No. 61/223,081, which was filed Jul. 6, 2009, U.S. Provisional Patent Application No. 61/223,084, which was filed Jul. 6, 2009, U.S. Provisional Patent Application No. 61/223,086, which was filed Jul. 6, 2009, U.S. Provisional Patent Application No. 61/223,088, which was filed Jul. 6, 2009, U.S. Provisional Patent Application No. 61/223,085, which was filed Jul. 6, 2009, U.S. Provisional Patent Application No. 61/223,405, which was filed Jul. 7, 2009, U.S. Provisional Patent Application No. 61/223,423, which was filed Jul. 7, 2009, U.S. Provisional Patent Application No. 61/223,425, which was filed Jul. 7, 2009, all of which are hereby incorporated herein by reference in their entireties.

### TECHNICAL FIELD OF THE DISCLOSURE

The present disclosure relates to microfluidic cytometry systems.

### BACKGROUND OF THE DISCLOSURE

Flow cytometry-based cell sorting was first introduced to the research community more than 20 years ago. It is a technology that has been widely applied in many areas of life science research, serving as a critical tool for those working in fields such as genetics, immunology, molecular biology and environmental science. Unlike bulk cell separation techniques such as immuno-panning or magnetic column separation, flow cytometry-based cell sorting instruments measure, classify and then sort individual cells or particles serially at rates of several thousand cells per second or higher. This rapid “one-by-one” processing of single cells has made flow cytometry a unique and valuable tool for extracting highly pure sub-populations of cells from otherwise heterogeneous cell suspensions.

Cells targeted for sorting are usually labeled in some manner with a fluorescent material. The fluorescent probes bound to a cell emit fluorescent light as the cell passes through a tightly focused, high intensity, light beam (typically a laser beam). A computer records emission intensities for each cell. These data are then used to classify each cell for specific sorting operations. Flow cytometry-based cell sorting has been successfully applied to hundreds of cell types, cell constituents and microorganisms, as well as many types of inorganic particles of comparable size.

Flow cytometers are also applied widely for rapidly analyzing heterogeneous cell suspensions to identify constituent sub-populations. Examples of the many applications where flow cytometry cell sorting is finding use include isolation of rare populations of immune system cells for AIDS research, isolation of genetically atypical cells for cancer research, isolation of specific chromosomes for genetic studies, and isolation of various species of microorganisms for environmental studies. For example, fluorescently labeled monoclonal antibodies are often used as “markers” to identify immune cells such as T lymphocytes and B lymphocytes, clinical laboratories routinely use this technology to count the number of “CD4 positive” T cells in HIV infected patients, and they also use this technology to identify cells associated with a variety of leukemia and lymphoma cancers.

Recently, two areas of interest are moving cell sorting towards clinical, patient care applications, rather than strictly research applications. First is the move away from chemical pharmaceutical development to the development of biopharmaceuticals. For example, the majority of new cancer therapies are bio-based. These include a class of antibody-based cancer therapeutics. Cytometry-based cell sorters can play a vital role in the identification, development, purification and, ultimately, production of these products.

Related to this is a move toward the use of cell replacement therapy for patient care. Much of the current interest in stem cells revolves around a new area of medicine often referred to as regenerative therapy or regenerative medicine. These therapies may often require that large numbers of relatively rare cells be isolated from sample patient tissue. For example, adult stem cells may be isolated from bone marrow and ultimately used as part of a re-infusion back into the patient from whom they were removed. Cytometry lends itself very well to such therapies.

There are two basic types of cell sorters in wide use today. They are the “droplet cell sorter” and the “fluid switching cell sorter.” The droplet cell sorter utilizes micro-droplets as containers to transport selected cells to a collection vessel. The micro-droplets are formed by coupling ultrasonic energy to a jetting stream. Droplets containing cells selected for sorting are then electrostatically steered to the desired location. This is a very efficient process, allowing as many as 90,000 cells per second to be sorted from a single stream, limited primarily by the frequency of droplet generation and the time required for illumination.

A detailed description of a prior art flow cytometry system is given in United States Published Patent Application No. US 2005/0112541 A1 to Durack et al.

Droplet cell sorters, however, are not particularly biosafe. Aerosols generated as part of the droplet formation process can carry biohazardous materials. Because of this, biosafe droplet cell sorters have been developed that are contained within a biosafety cabinet so that they may operate within an essentially closed environment. Unfortunately, this type of system does not lend itself to the sterility and operator protection required for routine sorting of patient samples in a clinical environment.

The second type of flow cytometry-based cell sorter is the fluid switching cell sorter. Most fluid switching cell sorters utilize a piezoelectric device to drive a mechanical system which diverts a segment of the flowing sample stream into a collection vessel. Compared to droplet cell sorters, fluid switching cell sorters have a lower maximum cell sorting rate due to the cycle time of the mechanical system used to divert the sample stream. This cycle time, the time between initial sample diversion and when stable non-sorted flow is restored, is typically significantly greater than the period of a droplet generator on a droplet cell sorter. This longer cycle time limits fluid switching cell sorters to processing rates of several hundred cells per second. For the same reason, the stream segment switched by a fluid cell sorter is usually at least ten times the volume of a single micro-drop from a droplet generator. This results in a correspondingly lower concentration of cells in the fluid switching sorter's collection vessel as compared to a droplet sorter's collection vessel.

Newer generation microfluidics technologies offer great promise for improving the efficiency of fluid switching devices and providing cell sorting capability on a chip similar in concept to an electronic integrated circuit. Many microfluidic systems have been demonstrated that can successfully sort cells from heterogeneous cell populations. They have the advantages of being completely self-contained, easy to sterilize, and can be manufactured on sufficient scales (with the resulting manufacturing efficiencies) to be considered a disposable part.

A generic microfluidic device is schematically illustrated in FIG. 1 and indicated generally at 10. The microfluidic device 10 comprises a substrate 12 having a fluid flow channel 14 formed therein by any convenient process as is known in the art. The substrate 12 may be formed from glass, plastic or any other convenient material, and may be substantially transparent or substantially transparent in a portion thereof. The substrate 12 further has three ports 16, 18 and 20 coupled thereto. Port 16 is an inlet port for a sheath fluid. Port 16 has a central axial passage that is in fluid communication with a fluid flow channel 22 that joins fluid flow channel 14 such that sheath fluid entering port 16 from an external supply (not shown) will enter fluid flow channel 22 and then flow into fluid flow channel 14. The sheath fluid supply may be attached to the port 16 by any convenient coupling mechanism as is known to those skilled in the art.

Port 18 also has a central axial passage that is in fluid communication with a fluid flow channel 14 through a sample injection tube 24. Sample injection tube 24 is positioned to be coaxial with the longitudinal axis of the fluid flow channel 14. Injection of a liquid sample of cells into port 18 while sheath fluid is being injected into port 16 will therefore result in the cells flowing through fluid flow channel 14 surrounded by the sheath fluid. The dimensions and configuration of the fluid flow channels 14 and 22, as well as the sample injection tube 24 are chosen so that the sheath/sample fluid will exhibit laminar flow as it travels through the device 10, as is known in the art. Port 20 is coupled to the terminal end of the fluid flow channel 14 so that the sheath/sample fluid may be removed from the microfluidic device 10.

While the sheath/sample fluid is flowing through the fluid flow channel 14, it may be analyzed using cytometry techniques by shining an illumination source through the substrate 12 and into the fluid flow channel 14 at some point between the sample injection tube 24 and the outlet port 20. Additionally, the microfluidic device 10 could be modified to provide for a cell sorting operation, as is known in the art.

Although basic microfluidic devices similar to that described hereinabove have been demonstrated to work well, there is a need in the prior art for improvements to cytometry systems employing microfluidic devices. The present invention is directed to meeting this need.

### SUMMARY OF THE DISCLOSURE

The present disclosure relates to microfluidic devices adapted for facilitating cytometry analysis of particles flowing therethrough. In certain embodiments, the microfluidic devices have onboard data storage capabilities. In certain other embodiments, the microfluidic devices have onboard anticoagulants. In certain other embodiments, the microfluidic devices have onboard test and control channels. In certain other embodiments, the microfluidic devices have integrated collection media. In certain other embodiments, the microfluidic devices have multiple onboard test channels. In certain other embodiments, the microfluidic devices have localized temperature control. In certain other embodiments, the microfluidic devices have anatomy simulating regions. In certain other embodiments, the microfluidic devices have complete assay capabilities. In certain other embodiments, the microfluidic devices have dissociable sections. In certain other embodiments, the microfluidic devices have means for performing functional assays.

In one embodiment, a microfluidic device is disclosed, comprising: a substrate, a microfluidic flow channel formed in said substrate, wherein said flow channel extends through a portion of said substrate adapted to facilitate cytometry analysis of cells flowing in said flow channel, and a data storage medium onboard said substrate, said data storage medium operative to store data relating to use of said microfluidic device.

In another embodiment, a method of detecting cells in a sample is disclosed, the method comprising the steps of: a) providing a microfluidic device, said microfluidic device comprising a substrate, a microfluidic flow channel formed in said substrate, wherein said flow channel extends through a portion of said substrate adapted to facilitate cytometry analysis of cells flowing in said flow channel, and a data storage medium onboard said substrate, said data storage medium operative to store data relating to use of said microfluidic device; b) performing a cytometry analysis of cells flowing in said flow channel; and c) recording data on said data storage medium.

In another embodiment, a microfluidic device is disclosed, comprising a substrate, a microfluidic flow channel formed in said substrate, wherein said flow channel extends through a portion of said substrate adapted to facilitate cytometry analysis of cells flowing in said flow channel, a sample well fluidically coupled to said flow channel, and an anticoagulant disposed in said sample well prior to introduction of a sample into said sample well.

In still other embodiments, a method of detecting cells in a sample is disclosed, the method comprising the steps of: a) providing a microfluidic device, said microfluidic device comprising a substrate, a first microfluidic flow channel formed in said substrate, wherein said first flow channel extends through a first portion of said substrate adapted to facilitate cytometry analysis of cells flowing in said first flow channel, and a second microfluidic flow channel formed in said substrate, wherein said second flow channel extends through a second portion of said substrate adapted to facilitate cytometry analysis of cells flowing in said second flow channel; b) placing a test sample in said first flow channel; c) performing a cytometry analysis of cells flowing in said first flow channel; d) placing a control sample in said second flow channel; and e) performing a cytometry analysis of cells flowing in said second flow channel.

In yet other embodiments, a method of detecting cells in a sample is disclosed, the method comprising the steps of: a) providing a microfluidic device, said microfluidic device comprising a substrate, a microfluidic flow channel formed in said substrate, wherein said flow channel extends through a portion of said substrate adapted to facilitate cytometry analysis of cells flowing in said flow channel, a first well fluidically coupled to said microfluidic flow channel, said first well containing a material at a first concentration, and a second well fluidically coupled to said microfluidic flow channel, said second well containing said material at a second concentration; b) placing a test sample in said flow channel; c) performing a cytometry analysis of cells flowing in said flow channel; d) causing a first portion of said cells to enter said first well; e) causing a second portion of said cells to enter said second well; f) measuring a response of said first portion of said cells to said first concentration; and g) measuring a response of said second portion of said cells to said second concentration.

In other embodiments, a method of detecting cells in a sample is disclosed, the method comprising the steps of: a) providing a microfluidic device, said microfluidic device comprising a substrate, a first microfluidic flow channel formed in said substrate, wherein said first flow channel extends through a first portion of said substrate adapted to facilitate cytometry analysis of cells flowing in said first flow channel, and a second microfluidic flow channel formed in said substrate, wherein said second flow channel extends through a second portion of said substrate adapted to facilitate cytometry analysis of cells flowing in said second flow channel; b) placing a first portion of a test sample in said first flow channel; c) performing a cytometry analysis of cells flowing in said first flow channel; d) placing a second portion of the test sample in said second flow channel; and e) performing a cytometry analysis of cells flowing in said second flow channel.

In yet other embodiments, a microfluidic device is disclosed, comprising a substrate having a first thermal conductivity, a microfluidic flow channel formed in said substrate, wherein said flow channel extends through a portion of said substrate adapted to facilitate cytometry analysis of cells flowing in said flow channel, and a pad formed onboard said substrate, said pad having a second thermal conductivity, wherein said first thermal conductivity is different than said second thermal conductivity.

In still other embodiments, a microfluidic device is disclosed, comprising a substrate, a microfluidic flow channel formed in said substrate, wherein said flow channel extends through a portion of said substrate adapted to facilitate cytometry analysis of cells flowing in said flow channel, and an anatomy simulating region disposed within said flow channel.

In other embodiments, a microfluidic device is disclosed, comprising a substrate, a microfluidic flow channel formed in said substrate, wherein said flow channel extends through a portion of said substrate adapted to facilitate cytometry analysis of cells flowing in said flow channel, a sample receiving well formed onboard said substrate and fluidically coupled to said flow channel, said sample well operative to receive a sample, and a sample preparation well formed onboard said substrate and fluidically coupled to said flow channel, said sample preparation well containing material operative to prepare said sample for cytometry analysis.

In other embodiments, a method of analyzing cells in a sample is disclosed, the method comprising the steps of: a) providing a microfluidic device, said microfluidic device comprising a substrate, a microfluidic flow channel formed in said substrate, wherein said flow channel extends through a portion of said substrate adapted to facilitate cytometry analysis of cells flowing in said flow channel, a sample receiving well formed onboard said substrate and fluidically coupled to said flow channel, said sample well operative to receive a sample, and a sample preparation well formed onboard said substrate and fluidically coupled to said flow channel, said sample preparation well containing material operative to prepare said sample for cytometry analysis; b) placing a sample in the sample receiving well; c) causing said sample to flow in said flow channel to said sample preparation well where said sample will react with said material; d) causing said sample to flow out of said sample preparation well and into said flow channel; and e) performing a cytometry analysis of sample flowing in said flow channel.

In other embodiments, a method of analyzing cells in a sample is disclosed, the method comprising the steps of: a) providing a microfluidic device, said microfluidic device comprising a substrate, a microfluidic flow channel formed in said substrate, wherein said flow channel extends through a portion of said substrate adapted to facilitate cytometry analysis of cells flowing in said flow channel, and a sample receiving well formed onboard said substrate and fluidically coupled to said flow channel, said sample well operative to receive a sample; b) placing a sample in the sample receiving well; and c) dissociating said sample receiving well from said substrate.

In still other embodiments, a microfluidic device is disclosed, comprising a substrate, a microfluidic flow channel formed in said substrate, wherein said flow channel extends through a portion of said substrate adapted to facilitate cytometry analysis of cells flowing in said flow channel, an inner preparation channel onboard said substrate, said inner preparation channel being fluidically coupled to said flow channel, and an outer preparation channel onboard said substrate, said outer preparation channel enclosing at least a portion of said inner preparation channel.

Other embodiments are also disclosed.

### BRIEF DESCRIPTION OF THE DRAWINGS

FIG. 1 is a perspective view of a prior art microfluidic device.

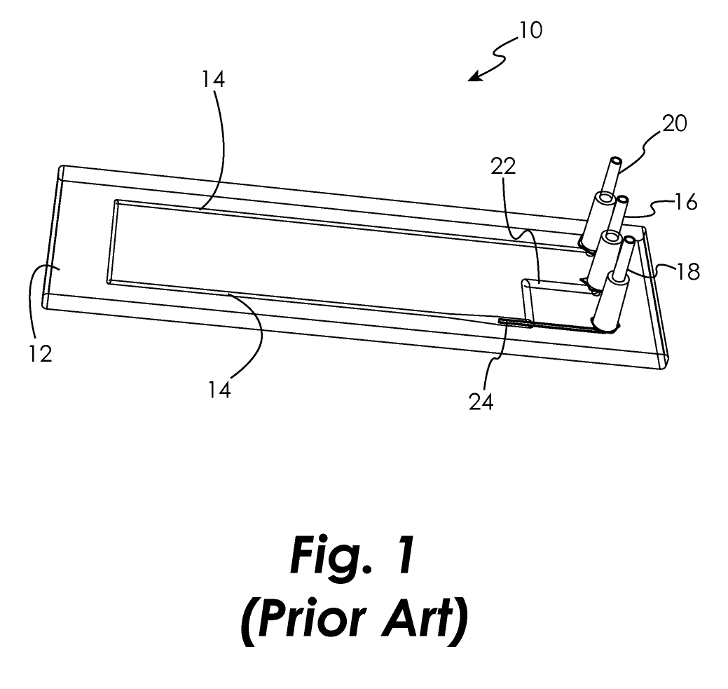

FIG. 2 is a schematic perspective view of a microfluidic device according to an embodiment of the present disclosure.

FIG. 3 is a schematic perspective view of a microfluidic device according to an embodiment of the present disclosure.

FIG. 4 is a schematic perspective view of a microfluidic device according to an embodiment of the present disclosure.

FIG. 5 is a schematic perspective view of a microfluidic device according to an embodiment of the present disclosure.

FIG. 6 is a schematic perspective view of a microfluidic device according to an embodiment of the present disclosure.

FIG. 7 is a schematic perspective view of a microfluidic device according to an embodiment of the present disclosure.

FIG. 8 is a schematic perspective view of a microfluidic device according to an embodiment of the present disclosure.

FIG. 9 is a schematic cross-sectional side view of a portion of a microfluidic device according to an embodiment of the present disclosure.

FIG. 10 is a schematic cross-sectional side view of a portion of a microfluidic device according to an embodiment of the present disclosure.

FIG. 11 is a schematic side cross-sectional view of a portion of a flow channel of a microfluidic device according to an embodiment of the present disclosure.

FIG. 12 is a schematic side cross-sectional view of a portion of a microfluidic device according to an embodiment of the present disclosure.

FIG. 13 is a schematic perspective view of a microfluidic device according to an embodiment of the present disclosure.

FIG. 14 is a schematic perspective view of a microfluidic device according to an embodiment of the present disclosure.

FIG. 15 is a schematic perspective view of a microfluidic device according to one embodiment of the present disclosure.

FIG. 16 is an enlarged front view section of the inner preparation channel and outer preparation channel of the device of FIG. 15.

### DESCRIPTION OF THE ILLUSTRATED EMBODIMENTS

For the purposes of promoting an understanding of the principles of the disclosure, reference will now be made to the embodiments illustrated in the drawings and specific language will be used to describe the same. It will nevertheless be understood that no limitation of the scope of the disclosure is thereby intended, such alterations and further modifications in the illustrated device, and such further applications of the principles of the disclosure as illustrated therein are contemplated as would normally occur to one skilled in the art to which the disclosure relates.

### Microfluidic Device Having Data Storage Capacity

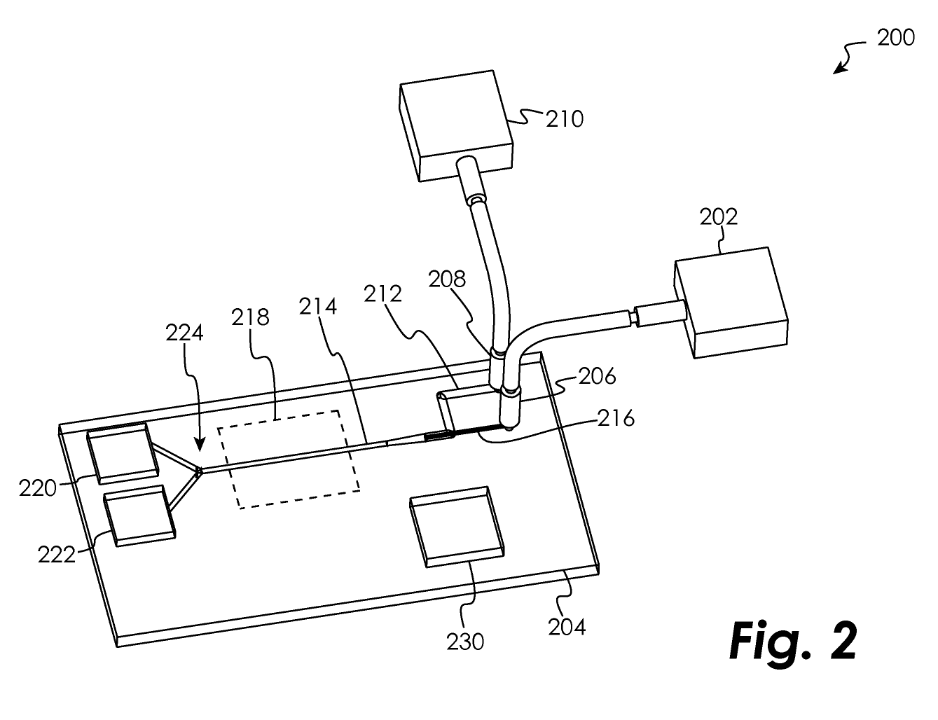

Certain embodiments of the present disclosure are generally directed to a system for the storage and retrieval of data on a microfluidic cytometry device, such as a cytometry chip. In FIG. 2, a system 200 is schematically illustrated in which cells coming from an external cell supply 202 are analyzed via cytometry using a microfluidic device formed onboard (i.e. on and/or in) substrate 204. As used herein, the term “onboard” is intended to encompass a structure that is carried by the substrate, whether that structure is on the substrate, in the substrate, or partially on and partially in the substrate. Cells from external supply 202 are input to the microfluidic device 200 through an input port 206. Port 208 is an inlet port for a sheath fluid from sheath fluid supply 210. Port 208 has a central axial passage that is in fluid communication with a fluid flow channel 212 such that sheath fluid entering port 208 from external supply 210 will enter fluid flow channel 212 and then flow into the main fluid flow channel 214. The sheath fluid supply 210 may be attached to the port 208 by any convenient coupling mechanism as is known to those skilled in the art. It is also possible that a system that does not require sheath flow can be employed.

Port 206 also has a central axial passage that is in fluid communication with a fluid flow channel 214 through a sample injection tube 216. Sample injection tube 216 is positioned to be coaxial with the longitudinal axis of the fluid flow channel 214. Injection of a liquid sample of cells from cell supply 202 into port 206 while sheath fluid is being injected into port 208 will therefore result in the cells flowing through fluid flow channel 214 surrounded by the sheath fluid. The dimensions and configuration of the fluid flow channels 214 and 212, as well as the sample injection tube 216 are chosen so that the sheath/sample fluid will exhibit laminar flow as it travels through the device 200, as is known in the art.

Cytometry analysis, possibly using a device external to the microfluidic device, may be performed in analysis section 218 (the specific operations that occur in analysis section 218 are not critical to the present disclosure). As a result of the analysis performed in section 218, the cells may optionally be sorted into different sample wells 220 or 222 based on differing characteristics of the cells. In certain embodiments, the sample wells 220, 222 have outlet ports (not shown) in fluid communication therewith in order to facilitate removal of the sorted sample from the wells. Sorting of cells may be accomplished by appropriate control of flow diverter 224.

In one embodiment, the flow diverter 224 is a piezoelectric device that can be actuated with an electric command signal in order to mechanically divert the flow through the sorting channel 214 into either the well 220 or the well 222, depending upon the position of the flow diverter 224. In other embodiments, flow diverter 224 is not a piezoelectric device, but instead can be, for example, an air bubble inserted from the wall to deflect the flow, a fluid deflector moved or actuated by a magnetic field or any other flow diverter or sorting gate as would occur to one of ordinary skill in the art.

In certain embodiments, cells may be sorted into different sample wells based on the intended future use for the cells. For example, cells having the same characteristics, or phenotype, may be sorted into one well where they are fixed for viewing, and sorted into another well where they are maintained in a viable state to undergo additional functional measurements. In other embodiments, the cells may be deposited into the wells based upon volume as opposed to a sorting method. For simplicity and ease of illustration, FIG. 2 schematically shows single channels extending between the components, areas or sections of device 200. However, it should be appreciated that the single channels may be representative of multiple cytometry channels and a variety of possible configurations of channels as would occur to one skilled in the art.

A data storage medium 230 is positioned onboard substrate 204 and may contain information pertaining to the sample and/or sheath fluid, along with any information about the status and origin of the sample 202 or the operations performed or to be performed on the sample. For example, the data storage medium 230 may include the medical history of a patient, the pathologist report, dates the sheath fluid was manufactured, dates the sample was processed, identification of the technician performing the operation, the results of the processing, or any data relevant to the sample or the test that may need to be archived for future retrieval.

In certain embodiments, the chip 200 may move among multiple stations in an automated processing environment. Each automated station or device may write information to the chip 200 that will be read at the next station. In this manner, information can be passed between the stations asynchronously, but still be correlated to the sample. In the case of a pathology testing laboratory, the chip may first be used for flow cytometry and then viewed by a medical professional, such as a pathologist, who writes a final report. This report can be written to the chip 200 as well. The data on the chip 200 guarantees that all records related to the history and processing of the sample remain physically correlated with the sample and available to any medical professional to view during or after a diagnostic process. Such chips 200 provide not only archival of the specimen but archival of the data as well.

The data storage medium 230 may comprise a readable and/or writeable medium, such as a hologram, a nonvolatile random access memory or the like, a writeable DVD element, a magnetic stripe, or other storage medium known in the art. The data storage medium enables a user, whether directly or with the aid of another device, to store information into the data storage medium 230 and to retrieve data that was previously stored on the data storage medium 230. The location of the data storage section 230 on the chip 200 may also be standardized, so that different types of automated equipment can read or write information to or from the data storage section 230.

### Microfluidic Device Having Onboard Anticoagulant

Certain embodiments of the present disclosure are generally directed to systems for the separation and analysis of a biological sample on a microfluidic device using cytometry (such as flow cytometry or image cytometry). In many applications, such devices are used to analyze the properties of biological samples. Certain types of samples, such as human blood, may begin to coagulate or clot after being removed from their natural environment inside the body and deposited into the sample container within the microfluidic device. In order to prevent clotting, an anticoagulant agent may be added to the sample to increase the amount of time that the sample will be viable for analysis by the microfluidic device. However, this adds complexity to the sampling process and increases the possibility of outside contaminants being introduced into the sample (or potentially harmful sample components being released into the outside environment). In order to eliminate the need for the user to manually add the anticoagulant, the anticoagulant may instead be added to the sampling container within the microfluidic device during the manufacturing process.

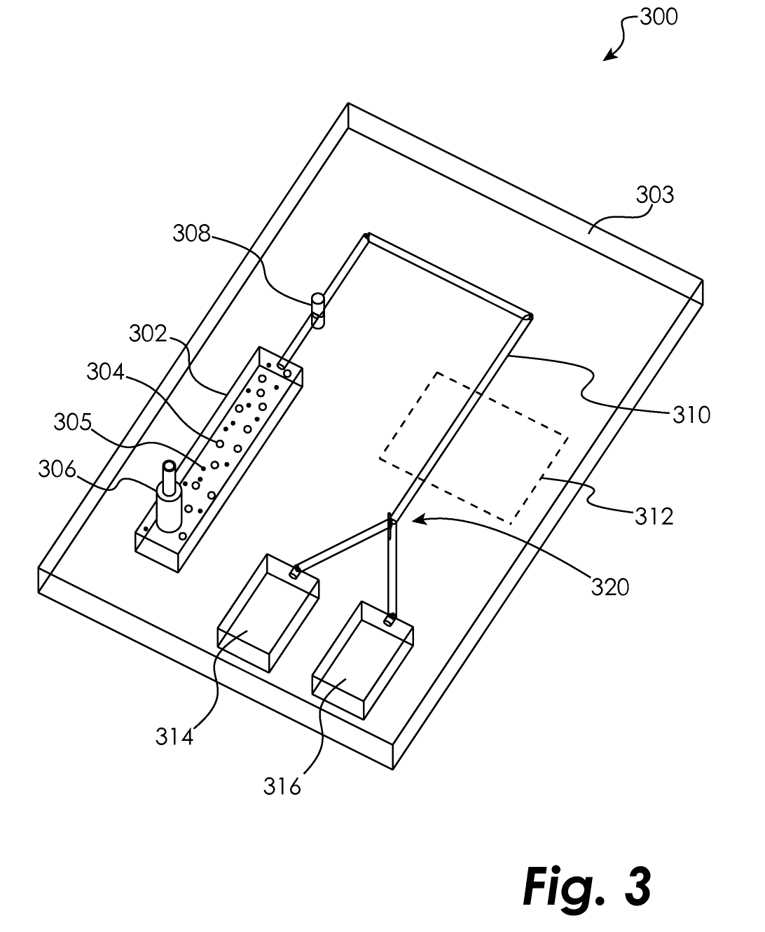

FIG. 3 schematically illustrates a microfluidic device 300 which is configured to analyze the biological components within a sample using cytometry. The device 300 includes a sample well 302 formed onboard a substrate 303 for receiving a sample 304 from an external source (not shown) via port 306. The sample well 302 contains an anticoagulant agent 305 that has been added during the manufacturing process, eliminating the need for the user to add it to the sample during use. For simplicity and ease of illustration, FIG. 3 shows single channels extending between the components, areas or sections of the microfluidic device 300. However, it should be appreciated that the single channels may be representative of multiple cytometry channels and a variety of possible configurations of channels as would occur to one skilled in the art.

Once the sample 304 has been added to the sample well 302 and mixed with the anticoagulant agent 305 already present therein, valve 308 is opened to allow the sample 304 to flow through flow channel 310 to cytometry analysis section 312 within device 300 for cytometric analysis and/or separation. The cytometric analysis may be performed in conjunction with an external device, and the specifics of the cytometric analysis are not critical to the present disclosure. For example, based upon the results in the cytometry analysis section 312, desired sample fluid may be diverted to extraction well 314 by appropriate control of flow diverter 320. Similarly, undesired cells in the sample may be diverted to the waste well 316 by appropriate control of flow diverter 320.

In one embodiment, the flow diverter 320 is a piezoelectric device that can be actuated with an electric command signal in order to mechanically divert the flow through the sorting channel 304 into either the outlet port 314 or the waste port 316, depending upon the position of the flow diverter 320. In other embodiments, flow diverter 320 is not a piezoelectric device, but instead can be, for example, an air bubble inserted from the wall to deflect the flow, a fluid deflector moved or actuated by a magnetic field or any other flow diverter or sorting gate as would occur to one of ordinary skill in the art.

The flow through channel 310 may be initiated by capillary action or other microfluidic pumping means known in the art. It shall be understood that the present disclosure contemplates that any type of biological or chemical sample that is prone to coagulation or clotting may be processed and/or analyzed using the disclosed device and method.

### Microfluidic Device Having Test and Control Channels

Certain embodiments of the present disclosure are generally directed to a system for providing at least one control cytometry channel and at least one test or experimental cytometry channel in parallel on a microfluidic device, thus enabling test and control assays to be conducted via a single microfluidic device. Performing the control assay in parallel with and substantially simultaneously with the test assay provides increased accuracy and precision of the cytometry testing. Additionally, the results from the multiple assays can be compared, averaged, or otherwise reviewed as a quality control step to provide a researcher or medical professional with increased assurance in the results of the cytometry testing.

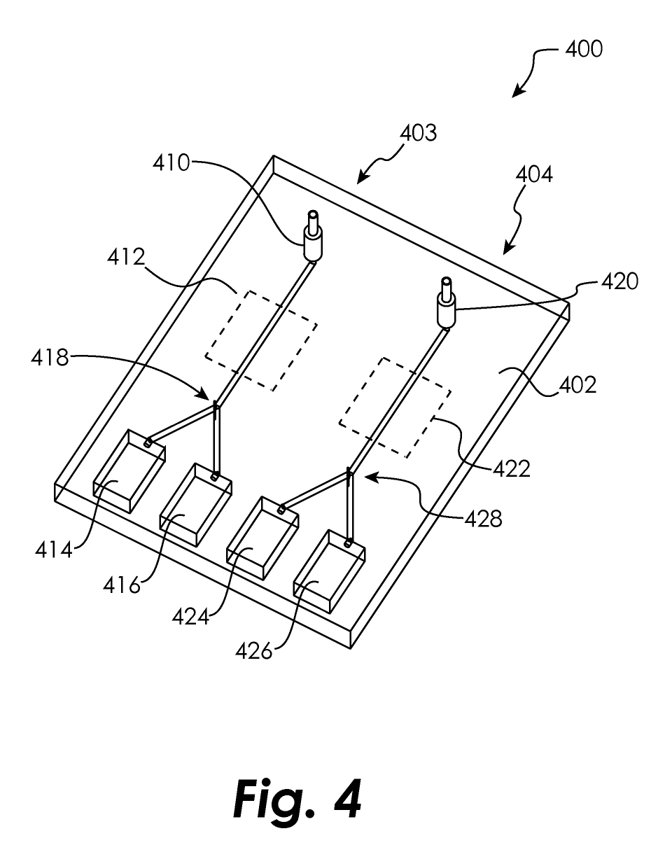

FIG. 4 illustrates a system 400 in which a microfluidic device formed onboard a substrate 402, provides for a test or experimental assay 403 in parallel with a control assay 404. As part of assay 403, material from a biological sample (not shown) is input to input port 410 and is analyzed via cytometry (such as, for example, flow cytometry or image cytometry) in analysis section 412 (the specific operations that occur in analysis section 412 are not critical to the present disclosure). According to the results of the analysis performed, the biological sample material may optionally be sorted into one or more different wells or chambers 414, 416. A first portion of the biological sample fluid may be diverted to well 414 by appropriate control of flow diverter 418. Similarly, a second portion of cells in the biological sample may be diverted to the well 416 by appropriate control of flow diverter 418.

In one embodiment, the flow diverter 418 is a piezoelectric device that can be actuated with an electric command signal in order to mechanically divert the flow through the sorting channel into either the well 414 or the well 416, depending upon the position of the flow diverter 418. In other embodiments, flow diverter 418 is not a piezoelectric device, but instead can be, for example, an air bubble inserted from the wall to deflect the flow, a fluid deflector moved or actuated by a magnetic field or any other flow diverter or sorting gate as would occur to one of ordinary skill in the art.

Additionally, as part of assay 404, material from a control sample (not shown) is input into input port 420 and is analyzed under the same conditions as the biological sample input at port 410 in analysis section 422 (the specific operations that occur in analysis section 422 are not critical to the present disclosure). According to the results of the analysis performed, the control sample material may optionally be sorted into one or more different wells or chambers 424, 426 by appropriate control of flow diverter 428. It should be appreciated that various components and sections shown on the substrate 402 as part of the cytometry assays are intended to show the operations of the cytometry process in a simple schematic and the cytometry components and sections on the device 400 can vary greatly as would occur to one of ordinary skill in the art.

Cells in the biological and control sample materials may be sorted into the different chambers 414, 416, 424 and 426 based on differing characteristics of the cells. Cells may be sorted into the different chambers 414, 416, 424 and 426 based on the intended future use for the cells. For example, cells having the same characteristics, or phenotype, may be sorted into one chamber where they are fixed for viewing and sorted into another chamber where they are maintained in a viable state to undergo additional functional measurements, or properly stored for use as part of a cell-based therapeutic procedure. As another example, desirable cells may be sorted into an extraction well or chamber and undesirable cells may be sorted into a waste well or chamber. Alternatively, the cells may be deposited into the chambers 414, 416, 424 and 426 based on volume as opposed to a sorting method. In certain embodiments, the sample wells 414, 416, 424 and 426 have outlet ports (not shown) in fluid communication therewith in order to facilitate removal of the sorted sample from the wells.

For simplicity, the illustration of FIG. 4 shows four chambers 414, 416, 424 and 426; however, it should be appreciated that the microfluidic device may include more or less than two chambers per assay as would occur to one of ordinary skill in the art. Additionally, the chambers 414, 416, 424 and 426 are shown as being horizontally aligned near the bottom of the substrate 402. However, it should be appreciated that the chambers 414, 416, 424 and 426, if present, may be positioned at other locations on the substrate 402 as would occur to one of ordinary skill in the art. Additionally, for simplicity and ease of illustration, FIG. 4 shows single channels extending between the components, areas or sections of substrate 402. However, it should be appreciated that the single channels may be representative of multiple cytometry channels and a variety of possible configurations of channels as would occur to one skilled in the art.

As illustrated, the test and control cytometry assay channels may be positioned in parallel on the substrate 402. However, it should be appreciated that the assay channels, including the analysis sections and the chambers, can be positioned otherwise on the substrate as would generally occur to one skilled in the art. Additionally, it is contemplated that the test and control assays 403 and 404 may occur substantially simultaneously or may occur consecutively in any order. In certain embodiments, the operations in analysis sections 412 and 422 are identical or substantially identical to ensure the accuracy and precision of the cytometry testing. As such, the control sample in assay 404 is tested under the same conditions as the biological sample in assay 403. In the illustrated embodiment, there is a single test assay 403 and a single control assay 404 incorporated into substrate 402. However, it should also be appreciated that substrate 402 may provide for additional test and/or control assays channels as would occur to one of ordinary skill in the art.

Providing the control assay in parallel with the test or experimental assay on the same microfluidic device and under the same conditions provides the researcher or medical professional with a known reference, via the results of the testing on the control material, with which to compare to the experimental assay results. Additionally, providing the control and experimental assays on the same microfluidic device removes the possibility of human error that might occur in testing the control sample and the experimental biological sample via different microfluidic devices at different times. In many situations, quality control assays are necessary or required to be performed to verify that the experimental assays are working properly. Providing the experimental and control assays on the same chip eliminates the need for the researcher or medical professional to separately run a control assay on a separate microfluidic device, reducing time and materials. Additionally, providing the experimental and control assays on the same chip may help to reduce error in situations where the reagents and sample processing are completed on the chip. Cell preparation for both the control sample and the biological sample can be completed simultaneously using the same lot of reagents which have been stored under the same conditions. Providing the experimental and control assays on the same chip allows for real time adjustments in the analysis procedure, such as total cells processed or process timing to affect both control and test samples in the same manner.

### Microfluidic Device Having Integrated Collection Media

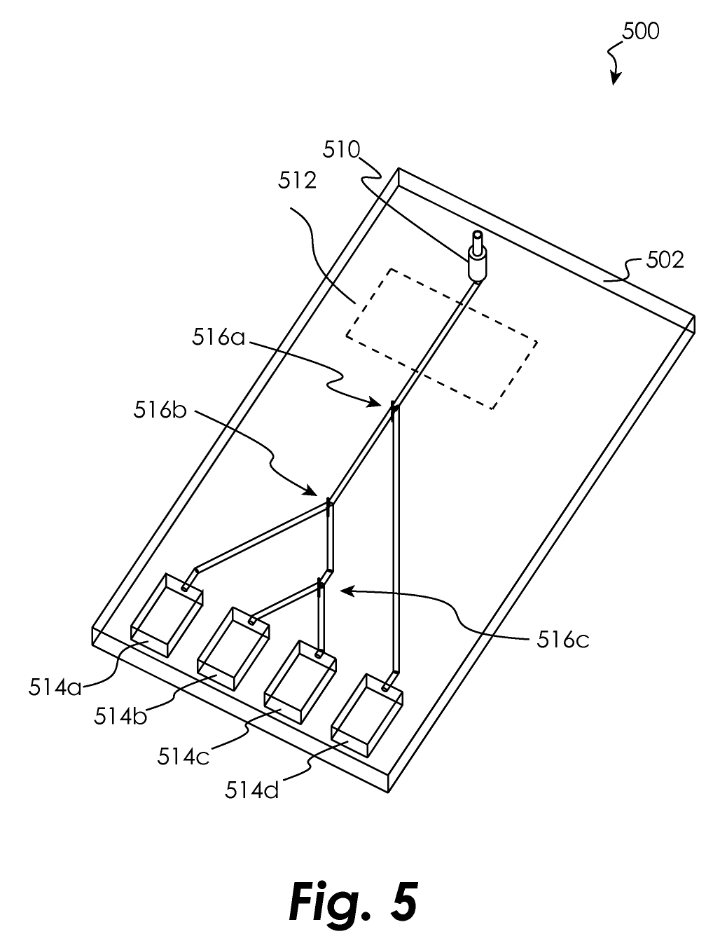

In certain embodiments, the present disclosure is generally directed to a system for the storage and preservation of cells on a microfluidic device 500 after the cells are analyzed and optionally sorted via the flow cytometry process described above. The storage and preservation of the cells may be accomplished via collection media integrated with the device 500. As schematically illustrated in FIG. 5, the cells come from a cell supply (not shown) and are input to an input port 510 formed on substrate 502. The cells input at port 510 are analyzed using cytometry in analysis section 512 (the specific operations that occur in analysis section 512 are not critical to the present disclosure). According to the results of the analysis performed, the cells may be sorted into different chambers 514 by appropriate control of flow diverters 516. In certain embodiments, the chambers 514 can include media such as reagents and/or other appropriate chemicals in order to maintain the integrity of the collected cells for post-analysis viewing or testing by a researcher or medical professional. For simplicity and ease of illustration, FIG. 5 shows single channels extending between the components, areas or sections of substrate 502. However, it should be appreciated that the single channels may be representative of multiple cytometry channels and a variety of possible configurations of channels as would occur to one skilled in the art.

The cells may be sorted into the different wells or chambers based on differing characteristics of the cells. Cells may be sorted into different wells or chambers based on the intended future use for the cells. For example, cells having the same characteristics, or phenotype, may be sorted into one well where they are fixed for viewing, and sorted into another well where they are maintained in a viable state to undergo additional functional measurements. Alternatively, the cells may be deposited into the chambers 514 based on volume or at random, as opposed to a characteristic-defined sorting method. In an example embodiment, cells determined in analysis section 512 to have a characteristic indicative of a cancer may be sorted into chambers 514a and 514b, and cells free of that cancer-indicating characteristic may be sorted into chambers 514c and 514d. As another example, cells determined to have a first characteristic may be sorted into chamber 514a, cells determined to have a second characteristic may be sorted into chamber 514b, cells determined to have a third characteristic may be sorted into chamber 514c, and cells determined to be free of the first, second and third characteristics may be sorted into chamber 514d. In other embodiments, all of the chambers 514 may receive cells having characteristics measured by analysis section 512.

In the illustrated embodiment there are four illustrated chambers; however, it should be appreciated that there may be more or less than four chambers as would generally occur to one skilled in the art. The chip having a plurality of chambers may be designed so that a predetermined amount of cells is sorted into each chamber. As an example, each chamber may receive one sorted cell. As another example, each chamber may receive up to ten sorted cells. As another example, each chamber may be capable of receiving up to the full amount of cells which are analyzed via the device 500, if necessary. For ease of illustration, the chambers 514 are illustrated as being horizontally aligned, but it should also be appreciated that the chambers may be positioned otherwise on the chip as would generally occur to one skilled in the art.

As mentioned above, the chambers may contain the necessary reagents and/or chemicals therein to fix the cells in their current state. In such a way, the each cell's visual appearance, or morphology, remains substantially in the same state as when the cell was sorted. This procedure, used routinely for microscope-based observation of cells, maintains the integrity of the sorted and isolated cells, substantially preventing the cells from breaking down and thus preserving the morphological characteristic(s) of the cells which dictated their sorting for later observation by a researcher or medical professional. In some embodiments, the reagents and/or chemicals maintain the cells in a natural, viable state so that they can be placed in culture or used for additional functional measurements. In some embodiments, the reagents and/or chemicals in the chambers may facilitate preparation of the cells for freezing, such as by freezing the entire device 500, for example. As an example, the entire chip, or a portion of the chip, could be placed in an automated cell cryogenic device. After the analysis is performed with respect to the cells on the chip, the researcher or medical professional may wish to view one or more of the cells having the characteristic(s) at issue under a microscope or similar device, or to run further tests or analysis on the cells. In certain embodiments, the chips may be prepackaged with the necessary reagents and/or chemicals in one or more of the chambers 514.

In some embodiments, the chambers may contain various concentrations of a material so that the response of the cell to the concentration of the material can be directly measured on the device. For example, in the course of validating a potential pharmaceutical it is often necessary to test the potential toxicity of the pharmaceutical at a variety of concentrations. To accommodate this, chips could be pre-loaded with a matrix of wells or chambers, each having a different concentration of the test pharmaceutical. Cells could be sorted into these wells or chambers. Optionally, other reagents could also be preloaded or automatically added from other chambers to facilitate direct reading of the cells' response in each well. As an example, such measurement can be done using an automated device such as a microwell plate reader.

### Microfluidic Device Having Multiple Assay Channels

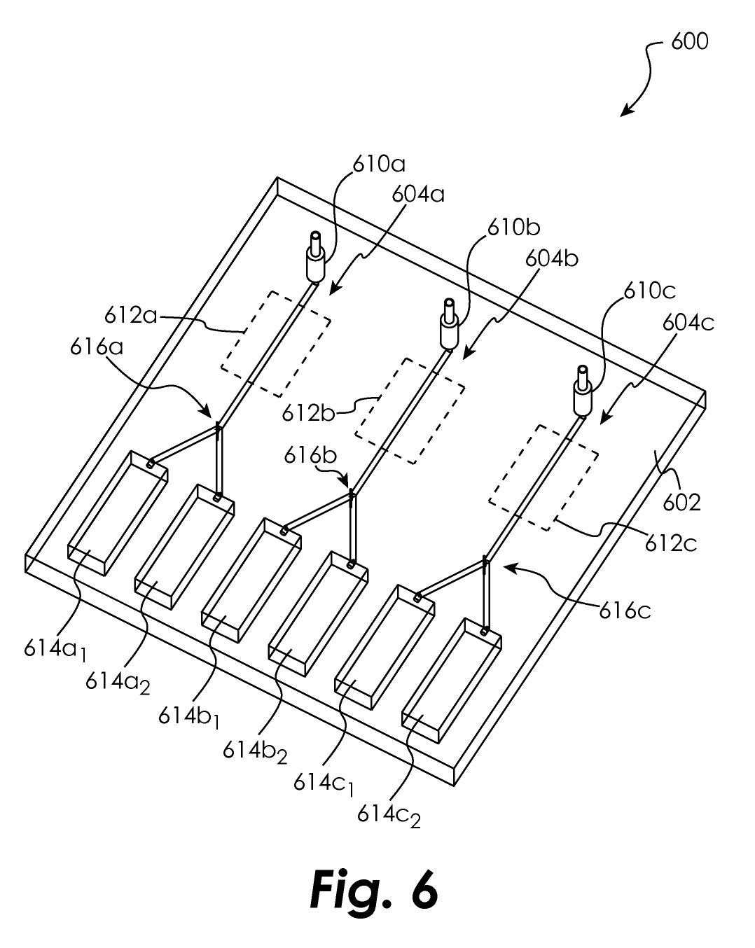

In certain embodiments, the present disclosure is generally directed to a system 600 including a plurality of identical cytometry channels (such as, for example, flow cytometry or image cytometry channels) on a microfluidic device, such as one formed on substrate 602, for providing multiple assays via a single microfluidic device. As schematically illustrated in FIG. 6, the cells for each cytometry analysis come from a single cell supply (not shown) and are supplied to input ports 610a, 610b and 610c and are analyzed via cytometry processes 604a, 604b and 604c in analysis sections 612a, 612b, and 612c respectively (the specific operations that occur in the analysis sections are not critical to the present disclosure). Providing for multiple cytometry analyses of cells from the same cell supply increases the accuracy and precision of the cytometry testing. The results from the multiple assays can be compared, averaged, or otherwise reviewed as a quality control step to provide a researcher or medical professional with increased assurance in the results of the cytometry testing.

As schematically illustrated, the multiple cytometry channels may be positioned in parallel on the chip. However, it should be appreciated that the channels, including the analysis sections and the chambers, can be positioned otherwise on the chip as would generally occur to one skilled in the art. Additionally, it is contemplated that the multiple assays via cytometry may occur substantially simultaneously or may occur consecutively in any order. It is also contemplated that the plurality of cytometry assays may be identical or substantially identical. In the illustrated embodiment, there are three cytometry analysis channels incorporated into chip 600. However, it should also be appreciated that chip 600 may provide for more or less than three assays as would occur to one skilled in the art.

According to the results of each analysis performed, the cells may optionally be sorted into different wells or chambers within chamber sets 614a, 614b, and 614c based on differing characteristics of the cells. Sample fluid may be diverted to well sets 614a, 614b, and 614c by appropriate control of flow diverters 616a, 616b and 616c, respectively.

In one embodiment, the flow diverters 616a, 616b and 616c comprise a piezoelectric device that can be actuated with an electric command signal in order to mechanically divert the flow through the sorting channels into either of the associated wells, depending upon the position of the flow diverters 616a, 616b and 616c. In other embodiments, flow diverters 616a, 616b and 616c are not a piezoelectric device, but instead can be, for example, an air bubble inserted from the wall to deflect the flow, a fluid deflector moved or actuated by a magnetic field or any other flow diverter or sorting gate as would occur to one of ordinary skill in the art.

Cells may be sorted into different wells or chambers based on the intended future use for the cells. For example, cells having the same characteristics, or phenotype, may be sorted into one well where they are fixed for viewing, and sorted into another well where they are maintained in a viable state to undergo additional functional measurements. Alternatively, the cells may be deposited into the wells or chambers based on volume or at random, as opposed to a characteristic-defined sorting method. In an example embodiment, cells determined in each analysis section 612a, 612b, and 612c to have a characteristic indicative of a cancer may be sorted into chambers 614a₁, 614b₁, and 614c₁ and cells free of that cancer-indicating characteristic may be sorted into chambers 614a₂, 614b₂, and 614c₂. In such embodiments, the researcher or medical professional might compare the results of the cytometry testing by comparing the quantity and characteristics of the sorted and isolated cells diverted into chambers 614a₁, 614b₁, and 614c₁. The chip may include means for physically diverting the cells into the chambers from the analysis section as is known in the art. In the illustrated embodiment there are two chambers leading from each analysis section; however, it should be appreciated that there may be more or less than two chambers from each analysis section as would generally occur to one skilled in the art. For ease of illustration, the chambers are schematically illustrated as being horizontally aligned, but it should also be appreciated that the chambers may be positioned otherwise on the chip as would generally occur to one skilled in the art. Alternatively, the cells may be caused to exit the chip 600 after the analysis is complete. For simplicity and ease of illustration, FIG. 6 shows single channels extending between the components, areas or sections of chip 600. However, it should be appreciated that the single channels may be representative of multiple cytometry channels and a variety of possible configurations of channels as would occur to one skilled in the art.

### Microfluidic Device Having Local Temperature Control

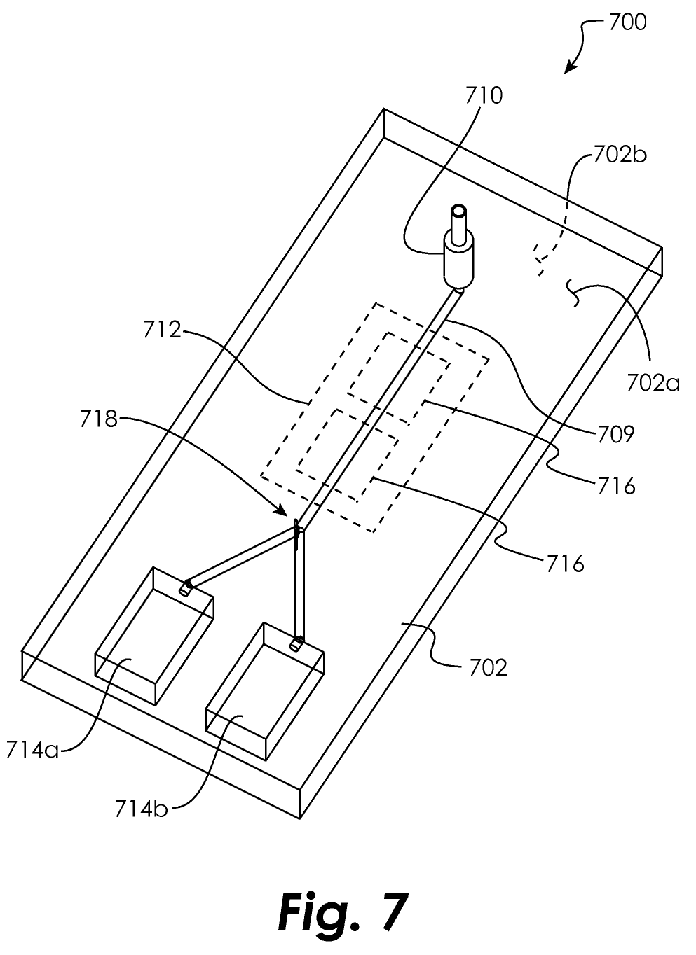

Certain embodiments of the present disclosure are generally directed to local temperature control for a microfluidic device. The systems contemplated by the present disclosure provide for dynamic temperature control at one or more specific locations on the microfluidic device. In embodiments involving cytometry (such as, for example, flow cytometry or image cytometry), the local temperature control may occur before, during and/or after the cells are analyzed and optionally sorted via the cytometry processes described herein (the specific operations that occur in the cytometry process are not critical to the present disclosure). FIGS. 7-10 illustrate just a few examples of possible local temperature control systems applied to or integrated with a microfluidic device.

FIG. 7 illustrates a system 700 for providing local temperature control with respect to two locations on a microfluidic device, such as the device formed on substrate 702 having a front side 702a and a back side 702b. The cells analyzed via chip 700 come from a cell supply (not shown) and are introduced at input port 710 and are analyzed via cytometry at one or more points in analysis section 712. For simplicity and ease of illustration, FIG. 7 shows a single flow channel 709 extending from input port 710 through analysis section 712. However, it should be appreciated that the single flow channel 709 may be representative of multiple cytometry channels and a variety of possible configurations of channels as would occur to one skilled in the art.

In the illustrated embodiment, system 700 includes temperature control devices 716 to control the temperature at particular localized segments of chip 700. In the particular illustrated embodiment, the temperature control devices 716 are positioned on back side 702b of chip 700 and control the temperature at specific locations within analysis section 712. However, it should be appreciated that the temperature control devices may be positioned elsewhere on the chip, including on front surface 702a and/or at various other locations throughout the cytometry process which occurs with respect to chip 700. Additionally, the illustrated embodiment shows two temperature control devices, however it is contemplated that there could be more or less than two temperature control devices as would occur to one skilled in the art.

In certain embodiments, chip 700 may include one or more wells or chambers 714 for temporary or permanent cell collection. In certain embodiments, the sample wells 714 have outlet ports (not shown) in fluid communication therewith in order to facilitate removal of the sorted sample from the wells. Sample fluid may be diverted to either of the wells 714 by appropriate control of flow diverter 718.

In one embodiment, the flow diverter 718 is a piezoelectric device that can be actuated with an electric command signal in order to mechanically divert the flow through the flow channel 709 into either well 714, depending upon the position of the flow diverter 718. In other embodiments, flow diverter 718 is not a piezoelectric device, but instead can be, for example, an air bubble inserted from the wall to deflect the flow, a fluid deflector moved or actuated by a magnetic field or any other flow diverter or sorting gate as would occur to one of ordinary skill in the art.

One or more temperature control devices may be positioned within, adjacent, or near the wells or chambers 714, or on the opposite side of the chip from the wells or chambers 714. In other embodiments, the one or more temperature control devices may be positioned adjacent to or on the opposite side of the chip from sections of the cytometry channels. In yet other embodiments, the one or more temperature control devices may be positioned at other possible and appropriate locations on the cytometry chip 700.

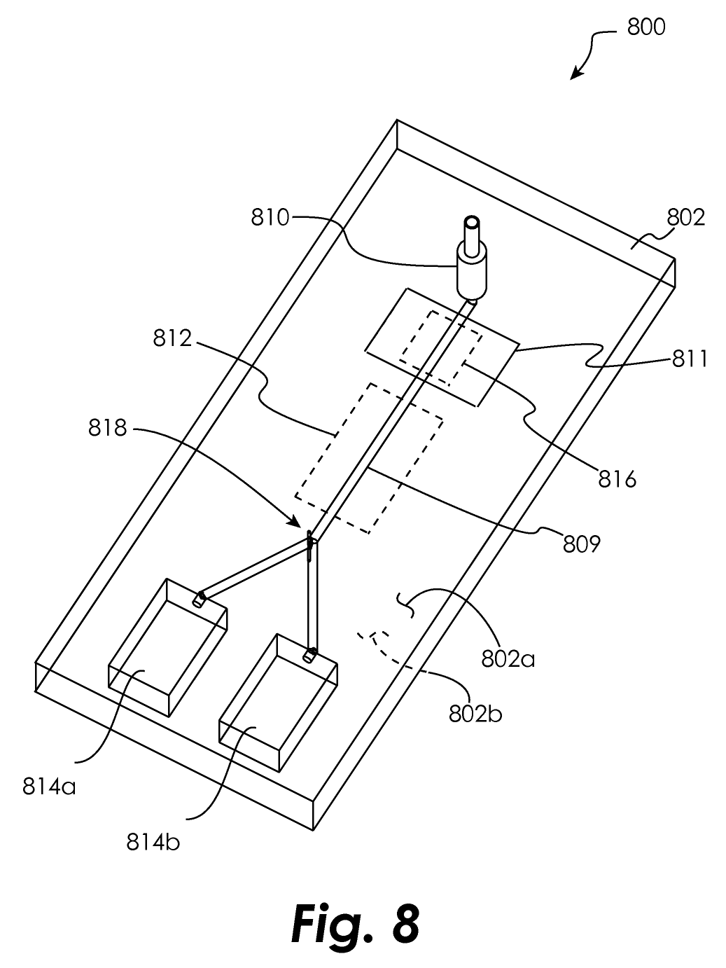

FIG. 8 illustrates another system 800 which provides for local temperature control on a microfluidic device, such as a device formed on substrate 802 having a front side 802a and a back side 802b. In the particular illustrated embodiment, chip 800 includes a cell preparation section 811 in which the cells are prepared for the cytometry analysis and the local temperature control occurs at section 811. The cells analyzed via chip 800 come from a cell supply (not shown) and are introduced at input port 810, travel to a cell preparation section 811, and are analyzed via cytometry at one or more points along analysis section 812. For simplicity and ease of illustration, FIG. 8 shows a single flow channel 809 extending from cell supply input port 810 and through analysis section 812. However, it should be appreciated that the single flow channel 809 may be representative of multiple flow cytometry tubes and a variety of possible configurations of channels as would occur to one skilled in the art.

As illustrated, chip 800 may include at least one temperature control device 816 positioned on back surface 802b to control the temperature at cell preparation section 811. Section 811 may be a well or a chamber configured to receive a raw cell sample prior to operation of the cytometry analysis. In certain embodiments, the cell sample preparation process requires increased temperature as opposed to the remaining portions of the cytometry process. Accordingly, it may be desirable to raise the temperature for a short period of time where the cell sample is being prepared and then lower the temperature of the cells prior to entry into the cytometry analysis section. As an example, preparation of the raw cell sample may involve the application of reagents and/or other chemicals to the sample, the reagents and/or chemicals being designed to appropriately prepare the sample for the cytometry analysis at certain temperatures.

Although FIG. 8 illustrates temperature control device 816 positioned on back surface 802b adjacent section 811, it should be appreciated that the temperature control device(s) may be positioned elsewhere on the chip, including on front surface 802a and/or at various other locations throughout the cytometry process which occurs with respect to chip 800. Additionally, the illustrated embodiment shows one temperature control device, however it is contemplated that there could be additional temperature control devices as would occur to one skilled in the art.

As one example, temperature control devices 716 and/or 816 may be solid-state heat transfer devices, such as Peltier heat-transfer devices as an example. However, many other temperature control apparatuses could be used in accordance with the present disclosure to control the temperature at localized regions on the cytometry chip. The temperature control devices may be attached or mounted to the chips via a variety of appropriate methods as would generally occur to one skilled in the art.

According to the results of the analyses performed in sections 712 and 812, the cells may optionally be sorted into different chambers 714 and 814, respectively, based on differing characteristics of the cells. In certain embodiments, the sample wells 814 have outlet ports (not shown) in fluid communication therewith in order to facilitate removal of the sorted sample from the wells. Sample fluid may be diverted to wells 814 by appropriate control of flow diverter 818.

In one embodiment, the flow diverter 818 is a piezoelectric device that can be actuated with an electric command signal in order to mechanically divert the flow through the flow channel 809 into either well 814, depending upon the position of the flow diverter 818. In other embodiments, flow diverter 818 is not a piezoelectric device, but instead can be, for example, an air bubble inserted from the wall to deflect the flow, a fluid deflector moved or actuated by a magnetic field or any other flow diverter or sorting gate as would occur to one of ordinary skill in the art.

Cells may be sorted into different wells or chambers based on the intended future use for the cells. For example, cells having the same characteristics, or phenotype, may be sorted into one well where they are fixed for viewing, and sorted into another well where they are maintained in a viable state to undergo additional functional measurements. Alternatively, the cells may be deposited into the wells or chambers based on volume or at random, as opposed to a characteristic-defined sorting method. In an example embodiment, cells determined in analysis section 712 (or 812) to have a characteristic indicative of a cancer may be sorted into chamber 714a (or 814a) and cells free of that cancer-indicating characteristic may be sorted into chamber 714b (or 814b). In the illustrated embodiments there are two chambers leading from each analysis section; however, it should be appreciated that there may be more or less than two chambers as would generally occur to one skilled in the art. For ease of illustration, the chambers are illustrated as being horizontally aligned, but it should also be appreciated that the chambers may be positioned otherwise on the chips as would generally occur to one skilled in the art. Alternatively, the cells may be caused to exit the chip 202 after the analysis is complete. For simplicity and ease of illustration, the figures show single channels extending between the components, areas or sections of the cytometry chips. However, it should be appreciated that the single channels may be representative of multiple cytometry channels and a variety of possible configurations of channels as would occur to one skilled in the art.

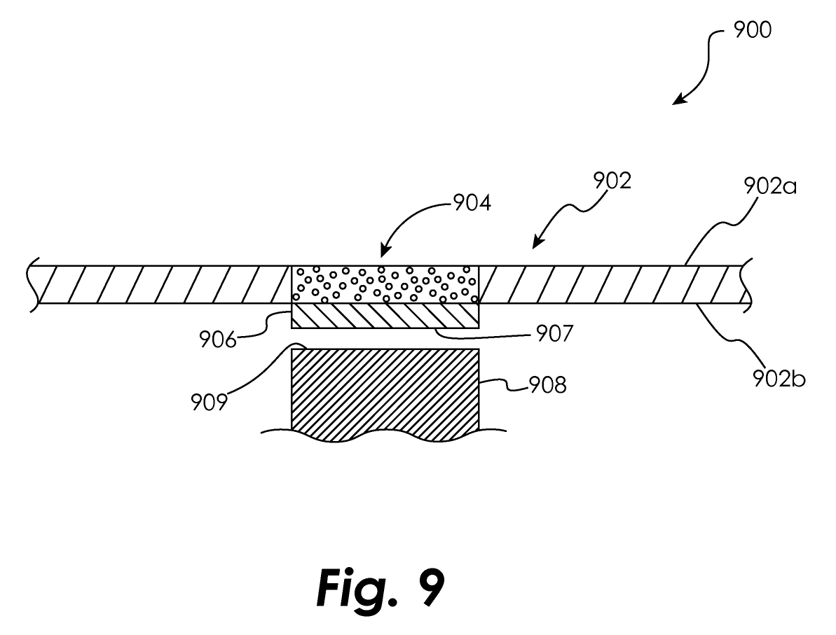

FIG. 9 illustrates another example embodiment of a local temperature control system of a microfluidic device. More specifically, FIG. 9 shows a cross-section of a microfluidic device, such as substrate 902 having front surface 902a and back surface 902b. Substrate 902 defines a well 904 configured to receive an amount of cell sample fluid involved in the cytometry analysis which occurs with respect to substrate 902. Adjacent well 904 is a pad 906 on back surface 902b of the chip. System 900 involves the application of a heating and/or cooling element 908 to pad 906 to provide localized temperature control to the material in well 904. In such embodiments, contact surface 909 of element 908 is configured to abut contact surface 907 of pad 906 to transfer temperature into well 904.

Pad 906 may be composed of any appropriate material capable of transferring a heating or cooling effect from element 908 into well 904. In certain embodiments, pad 906 may be an integrally molded portion of the chip. In other embodiments, pad 906 may be mounted or attached to the back surface of the chip via an appropriate method as would occur to one skilled in the art. In certain embodiments, pad 906 comprises a layer of aluminum. Additionally, heating and/or cooling element 908 may be any appropriate temperature control element as would occur to one skilled in the art. Although element 908 is illustrated as a single element, it should be appreciated that element 908 may be composed of multiple temperature control elements configured to contact pad 9906. Further, it is contemplated that a plurality of element and pad combinations may be incorporated into the cytometry process occurring with respect to substrate 902 and at a variety of different possible locations within the process as would occur to one skilled in the art. In such embodiments, the present disclosure contemplates that the different element and pad combinations may be adjusted to different localized temperatures and may be of differing sizes. In some embodiments, the substrate 902 fits within a fixture or other guide means that ensure alignment between the pad 906 and the element 908.

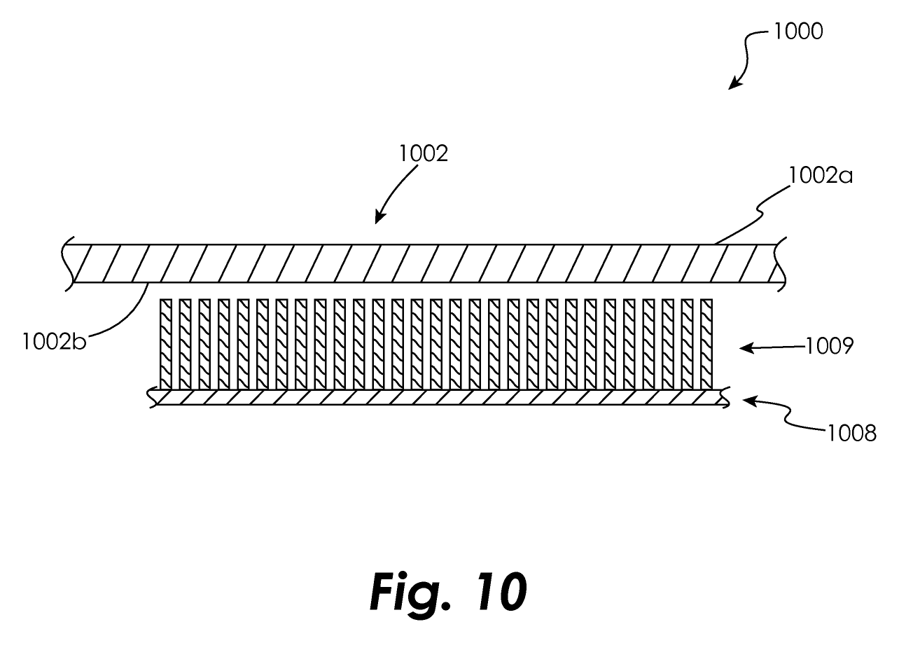

FIG. 10 illustrates yet another local temperature control system 1000. System 1000 involves the application of a heating and/or cooling element 1008 to a microfluidic device, such as one formed on substrate 1002 having a front surface 1002a and a back surface 1002b, to control the temperature at localized regions of the device. Element 1008 includes a plurality of prongs, needles or fingers 1009, the temperature of each finger being individually controlled. As such, setting the temperatures of different groups of needles which contact specific regions of the chip allows for localized temperature control along the chip. In certain embodiments, element 1008 is substantially as large as substrate 1002, such that the temperature at all areas of the chip may be controlled. In other embodiments, element 1008 is smaller than substrate 1002 and can be positioned at different locations along substrate 1002 to provide localized temperature control at the different locations. In certain embodiments, substrate 1002 defines one or more wells (not shown) configured to receive an amount of cell sample fluid involved in the cytometry analysis and has pads positioned on back surface 1002b aligned with each of the wells. In such embodiments, the tips of the fingers contact the pads which transmit the heating or cooling effect into the wells to provide localized temperature control to the particular amounts of cell sample with the wells.

Local temperature control on a microfluidic device analyzing cells is important at least because the temperature of a cell may be directly related to the metabolic activity of the cell, and the speed and/or quality of a reaction occurring with respect to the cell as part of the cytometry analysis. In certain situations it is desirable to perform a functional assay on cells which are alive and which require temperature control to perform properly under the assay. To that end, certain cells may stop functioning at certain temperatures and thus local temperature control may be used to maintain the functioning of the cells within the assay. In certain embodiments, increasing the temperature of the cell promotes the uptake of a reagent or speeds up the production of a metabolite by the cell, while decreasing the temperature of the cell functions to slow or stop the metabolism of the cell. In some embodiments, lowering the temperature of the cell may occur in conjunction with cryogenic freezing of the cell.

The local temperature control systems contemplated by the present disclosure may also provide for relatively quick, dynamic temperature changes. As an example, the systems may be capable of providing for a temperature increase for a specific amount of time followed by a relatively quick temperature decrease as necessary for the cytometry process, and vice versa. Additionally, in certain embodiments the chips may be composed of a plastic material operable to insulate against temperature transfer between different regions of the chip. As such, the local temperature control systems can allow for different regions of the chip to be at different temperatures simultaneously. This result is enhanced by the use of contact pads having a higher thermal conductivity than the material from which the substrate is constructed, such as contact pads of aluminum, placed on the surface of the substrate in areas where local temperature control is desired.

In certain embodiments, it is desirable to view or provide imaging of cells before or after the cytometry analysis. Cytometry chips may include wells or chambers to collect a portion of the cell sample for observation or imaging by a researcher or medical professional. Controlling the temperature of the isolated cells can be integral to such observation or imaging. Accordingly, in addition to the example embodiments discussed above, the localized temperature at a cell observation well or chamber can also be controlled via one or more of the local temperature control systems contemplated herein.

### Microfluidic Device Having Anatomy Simulating Channels

Certain embodiments of the present disclosure are generally directed to a microfluidic device, such as a cytometry chip (such as, for example, flow cytometry or image cytometry), having one or more anatomy simulating channels, wells and/or chambers. The anatomy simulating components may be positioned at various locations on the chip, and thus the cells may encounter the anatomy simulating components at various locations throughout the cytometry process. As an example, the cells may flow through anatomy simulating channel(s) before, during and/or after the cells are analyzed and optionally sorted via the cytometry process described herein (the specific operations that occur in the cytometry process are not critical to the present disclosure). Providing one or more anatomy simulating components allows the researcher or medical professional to observe the cells before, during and after the interaction of the cells with the simulated anatomy.

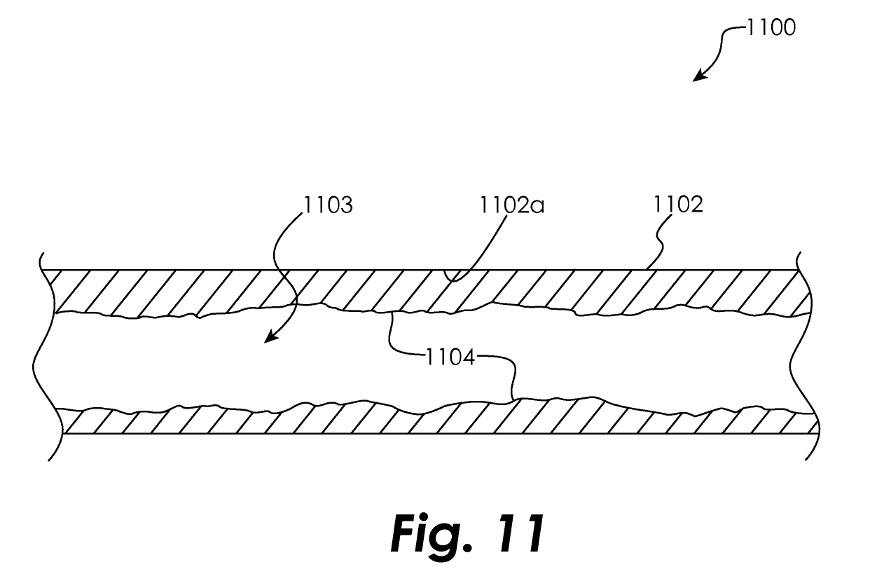

FIG. 11 illustrates a flow channel 1100 on a microfluidic device, such as a cytometry chip, having anatomy simulating features. Channel 1100 is defined by a cylindrical channel wall 1102. However, it should be appreciated that the channel may be shaped and configured otherwise, such as square or rectangular in cross-section as examples. Wall 1102 has an inside surface 1102a to which anatomy simulating material 1104 is applied. The introduction of the anatomy simulating material to the interior of the channel provides the researcher or medical professional with the ability to observe, image and/or analyze the cells in an environment mimicking their natural environment in an internal bodily passageway.

In certain embodiments, material 1104 may be native tissue material from the anatomical area which is being simulated. In other embodiments, material 1104 may be reconstructed from other material to simulate the native tissue material. In certain embodiments, the anatomy simulating material may be grown in the particular chip component by placing cells of the particular anatomical area to be simulated in the component on the chip, incubating the chip in an incubator, and growing the cells to create the anatomy simulating material.

Cells flowing through the interior 1103 of channel 1100 encounter material 1104 as they would encounter native material when flowing through passageways in the body. The interior of the channel may be designed to simulate the interior of a variety of possible internal bodily passageways, such as blood vessels including arteries and veins, urinary tracts, and portions of the alimentary canal including the intestine, the esophagus, and the colon. In other embodiments, the interior of the channel may be designed to simulate the interior of a variety of possible internal bodily organs.

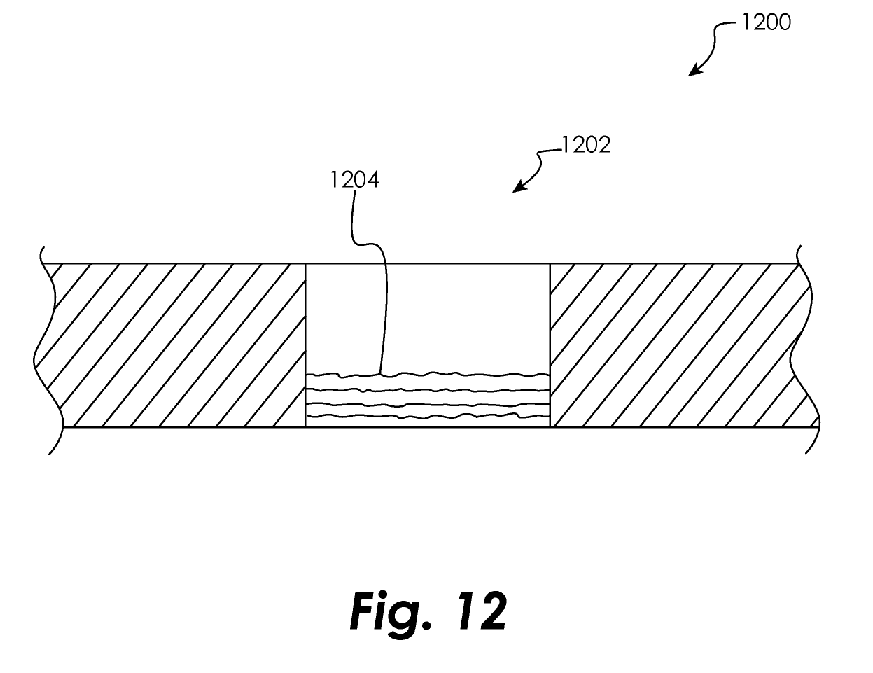

In other embodiments, a chemical technique can be used to mimic the natural internal bodily area. The chemical technique can include the application of chemicals and/or other materials to simulate the natural bodily environment. The chemical technique can be applied at one or more locations on a cytometry chip, including channels, wells and/or chambers. Additionally, the chemical technique can be applied at one or more stages of the cytometry process, including before, during and/or after the cells are analyzed via the cytometry analysis. FIG. 12 illustrates an example embodiment of application of a chemical technique. There is shown in FIG. 12 a portion of a flow cytometry chip 1200 defining a well 1202. Well 1202 is configured to receive an amount of cell sample fluid flow as part of the cytometry process. An amount of chemical 1204 is placed in well 1202, the chemical being designed to adjust the pH of the well environment to that of the native environment to be simulated. This provides the researcher or medical professional with the ability to observe, image and/or analyze the cells in an environment mimicking their natural environment in an internal bodily passageway, organ or other internal bodily location.

The anatomy simulating component(s) and/or the application of the chemical technique allow the researcher or medical professional to observe and assess how interaction with the simulated anatomy affects the cells. Additionally, the researcher or medical professional may apply a chemical, such as a pharmaceutical, to the sample to observe and assess how the chemical affects the interaction of the cells with the simulated anatomy. Further, the procedures described above provide the researcher or medical professional with the ability to observe and assess how the cells penetrate the simulated anatomy and the impact of such penetration on both. Also, the researcher or medical professional may conduct further observation, testing or analysis on both the cell sample passing through simulated anatomy or the simulated anatomy material following the controlled interaction therebetween. It should be appreciated that the references to cells and cell samples (for simplicity) contemplates the introduction of other material (alone or in combination), such as organisms, particles, and viruses, as non-limiting examples.

The anatomy simulating components contemplated by the present disclosure may take any convenient physical form. As examples, one or more of the components may be formed in the surface of the microfluidic device and may be open or may include a cover. The cover can be glued in place, snapped in place with resilient members that engage the device, slid in place under guides that extend from the surface of the device, or any other convenient means as would occur to one of ordinary skill in the art. As another example, one or more of the components may be closed. The above examples are intended to be only non-limiting examples of many possible configurations.

### Microfluidic Device Having Complete Assay Capabilities

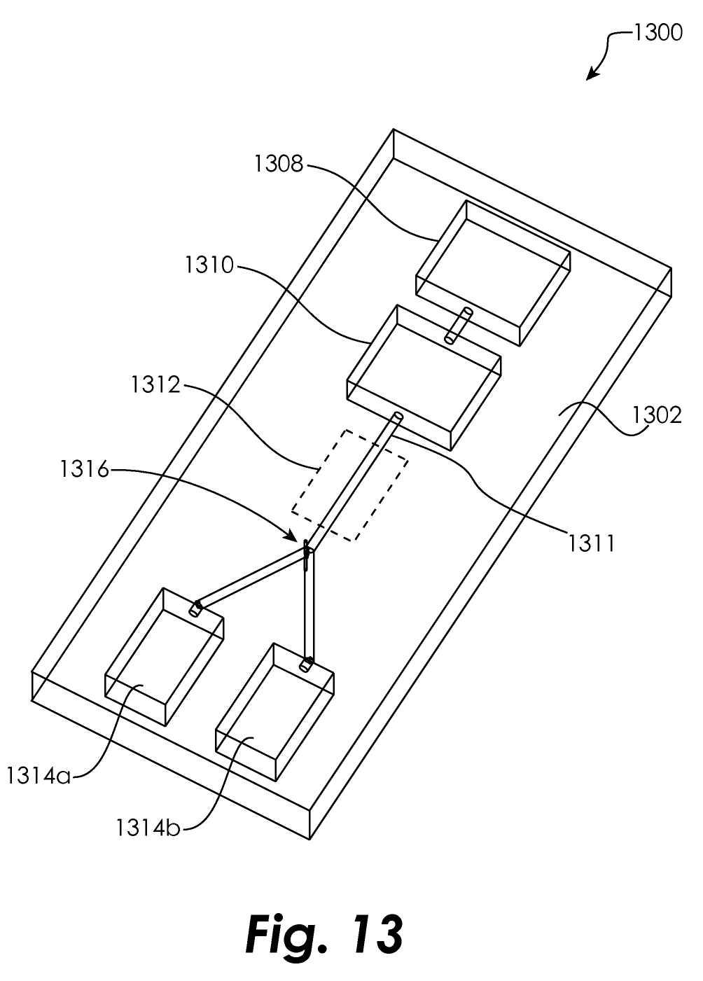

Certain embodiments of the present disclosure are generally directed to a system for providing complete cytometry assay capabilities with respect to a single microfluidic device, such as a cytometry chip. The raw (solid or fluid) sample to be analyzed may be placed directly on the chip, with the capability to prepare the sample for the cytometry analysis being incorporated into the chip. In certain embodiments, the cytometry assay includes a flow cytometry or image cytometry analysis. As schematically illustrated in FIG. 13, system 1300 includes raw sample (not shown) being placed into chamber or well 1308 on substrate 1302, prepared for flow cytometry analysis at section 1310, and analyzed via cytometry at analysis section 1312 (the specific operations that occur in analysis section 1312 are not critical to the present disclosure). In some embodiments, the raw sample may be a fluid sample such as blood or urine as non-limiting examples.

Preparation of the raw sample at section 1310 involves the application of reagents and/or other chemicals to the raw sample, the reagents and/or chemicals being designed to appropriately prepare the sample for the cytometry analysis. In certain embodiments, section 1310 comprises a well or chamber, and the reagents and/or chemicals are placed in the well or chamber 1310 prior to delivery of the raw sample to that well or chamber. Additionally, the reagents and/or chemicals may be stored on the chip in a dried format, such as by freeze-drying the reagents and/or chemicals into a lyophilized format as an example. However, it should be appreciated that the reagents and/or chemicals may be stored on the chip in other formats as would occur to one skilled in the art.

According to the results of the cytometry analysis performed, the cells may optionally be sorted into different wells or chambers 1314 based on differing characteristics of the cells. In certain embodiments, the sample wells 1314 have outlet ports (not shown) in fluid communication therewith in order to facilitate removal of the sorted sample from the wells. Sample fluid may be diverted to wells 1314 by appropriate control of flow diverter 1316.

In one embodiment, the flow diverter 1316 is a piezoelectric device that can be actuated with an electric command signal in order to mechanically divert the flow through the sorting channel 1311 into either of the wells 1314, depending upon the position of the flow diverter 1316. In other embodiments, flow diverter 1316 is not a piezoelectric device, but instead can be, for example, an air bubble inserted from the wall to deflect the flow, a fluid deflector moved or actuated by a magnetic field or any other flow diverter or sorting gate as would occur to one of ordinary skill in the art.

Cells may be sorted into different wells or chambers based on the intended future use for the cells. For example, cells having the same characteristics, or phenotype, may be sorted into one well where they are fixed for viewing, and sorted into another well where they are maintained in a viable state to undergo additional functional measurements. Alternatively, the cells may be deposited into the wells or chambers based on volume or at random, as opposed to a characteristic-defined sorting method. In an example embodiment, cells determined in analysis section 1312 to have a characteristic indicative of a cancer may be sorted into chamber 1314a and cells free of that cancer-indicating characteristic may be sorted into chamber 1314b. The chip 1300 may include means for physically diverting the cells into the chambers from the analysis section as is known in the art. In the illustrated embodiment there are two chambers leading from the analysis section; however, it should be appreciated that there may be more or less than two chambers as would generally occur to one skilled in the art. For ease of illustration, the chambers are illustrated as being horizontally aligned, but it should also be appreciated that the chambers may be positioned otherwise on the chip as would generally occur to one skilled in the art. Alternatively, the cells may be caused to exit the chip 1300 after the analysis is complete. Additionally, for simplicity and ease of illustration, FIG. 13 shows single channels extending the components, areas or sections of chip 1300. However, it should be appreciated that the single channels may be representative of multiple cytometry channels and a variety of possible configurations of channels as would occur to one skilled in the art.

In certain embodiments, chip 1300 may be designed for individual home use. In such embodiments, an individual can deposit the raw sample into a well on the chip. In an example situation, the raw sample is blood and the individual may (themselves or through use of another medical device) prick one of their fingers to expose a blood sample and deposit the blood sample into the well 1308 on the chip 1300. The raw sample may travel from its original placement to preparation section 1310 and from preparation section 1310 to analysis section 1312 via osmotic pumping or another appropriate method as would generally occur to one skilled in the art. After the raw sample is deposited onto the chip 1300 and prepared for the cytometry process at section 1310, the chip 1300 may be placed in a cytometry analysis machine in the individual's home and the cytometry analysis conducted. The machine may also be designed to provide the results of the cytometry testing to the individual. In such a way, the complete cytometry assay occurs without the need for intervention by a researcher or medical professional. After the analysis is complete, the chip 1300 may be disposed of, saved for later reference, or transferred to a researcher or medical professional for further processing. However, it should be appreciated that the complete cytometry assay with respect to chip 1300 may also occur in a medical facility via a researcher or medical professional.

### Microfluidic Device Having Dissociable Section

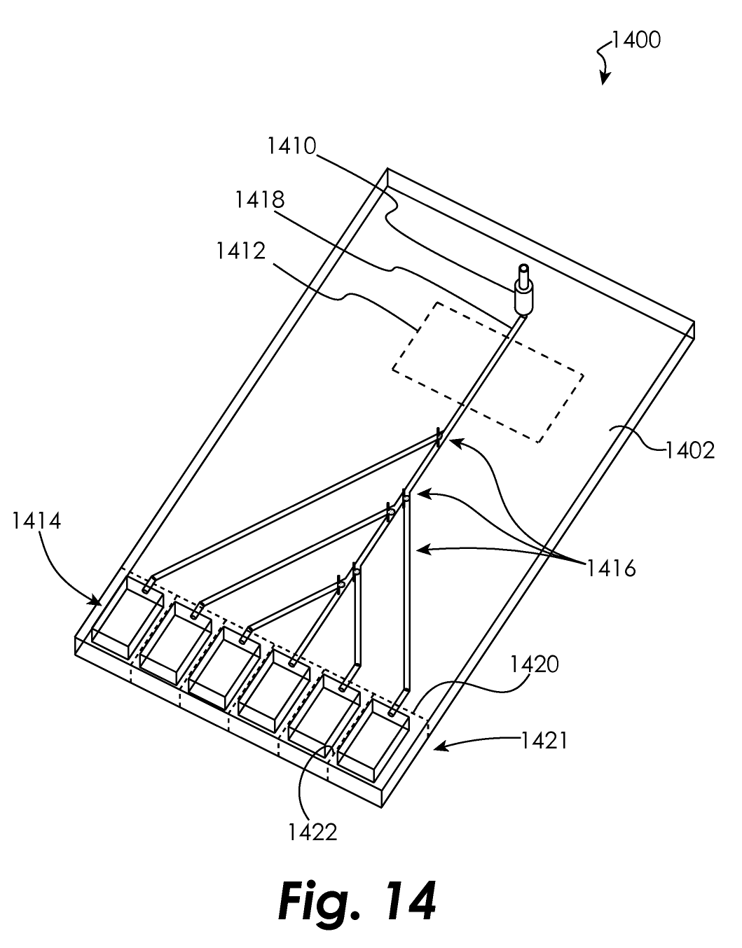

Certain embodiments of the present disclosure are generally directed to a microfluidic device having one or more dissociable sections for the storage, preservation and/or transport of cells on the dissociable section of the microfluidic device. As schematically illustrated in FIG. 14, system 1400 provides for a cytometry analysis (such as flow cytometry or image cytometry, as examples) of cells on a microfluidic device, such as one formed on substrate 1402, and sorting of the cells after the cells are analyzed via the cytometry process. In certain embodiments, the section may be dissociated from the chip 1400 either before or after freezing of the cells occurs.

As illustrated in FIG. 14, the cells come from a cell supply (not shown) and are input to input port 1410 and are analyzed in analysis section 1412 (the specific operations that occur in analysis section 1412 are not critical to the present disclosure). According to the results of the analysis performed, the cells may be sorted into different chambers 1414. In certain embodiments, the sample wells 1414 have outlet ports (not shown) in fluid communication therewith in order to facilitate removal of the sorted sample from the wells. Sample fluid may be diverted to wells 1414 by appropriate control of flow diverters 1416.

In one embodiment, the flow diverters 1416 are a piezoelectric devices that can be actuated with an electric command signal in order to mechanically divert the flow through the flow channel 1418 into any of the wells 1414, depending upon the positions of the flow diverters 1416. In other embodiments, flow diverters 1416 are not a piezoelectric device, but instead can be, for example, an air bubble inserted from the wall to deflect the flow, a fluid deflector moved or actuated by a magnetic field or any other flow diverter or sorting gate as would occur to one of ordinary skill in the art.

In certain embodiments, the chambers 1414 can include media such as reagents and/or other appropriate chemicals in order to prepare the cells for freezing and/or maintain the integrity of the collected cells for post-analysis viewing or testing by a researcher or medical professional. For simplicity and ease of illustration, FIG. 14 shows single channels extending between the components, areas or sections of chip 1400. However, it should be appreciated that the single channels may be representative of multiple cytometry channels and a variety of possible configurations of channels as would occur to one skilled in the art.

The cells may be sorted into the different wells or chambers 1414 based on differing characteristics of the cells. Cells may be sorted into different wells or chambers 1414 based on the intended future use for the cells. For example, cells having the same characteristics, or phenotype, may be sorted into one well where they are fixed for viewing, and sorted into another well where they are maintained in a viable state to undergo additional functional measurements. Alternatively, the cells may be deposited into the chambers 1414 based on volume or at random, as opposed to a characteristic-defined sorting method. Alternatively, some of the cells may be caused to exit chip 1400 after the cytometry analysis is complete.

In the illustrated embodiment there are six illustrated chambers 1414; however, it should be appreciated that there may be more or less than six chambers as would occur to one of ordinary skill in the art. The chip 1400 may be designed so that a predetermined amount of cells is sorted into each chamber 1414. As an example, each chamber 1414 may receive one sorted cell. As another example, each chamber 1414 may receive up to ten sorted cells. As another example, each chamber 1414 may be capable of receiving up to the full amount of cells which are analyzed via the chip 1400, if necessary. For ease of illustration, the chambers 1414 are illustrated as being horizontally aligned near the bottom of the chip 1400, but it should also be appreciated that the chambers may be positioned elsewhere on the chip as would occur to of ordinary skill in the art.

The chip 1400 includes a division line 1420 so that bottom section 1421 of the substrate 1402 may be dissociated from the remainder of the substrate 1402. In certain embodiments, after the cytometry analysis is complete, chip 1400 is placed in an automated cell cryogenic device and thus the cells sorted into the chambers 1414 are frozen. After the cells are frozen, the bottom section 1421 may be dissociated from chip 1400. Freezing of the cells in chambers 1414 prior to dissociating section 1421 may enhance the sterilization of the chambers 1414 and cells contained therein at least because the channels leading from analysis section 1412 and in communication with the chambers are frozen along with the chambers and the cells. In other embodiments, bottom section 1421 may be dissociated from the remainder of substrate 1402 prior to freezing of the cells, with just the dissociated section 1421 being placed in the automated cell cryogenic device. In addition to or in lieu of the freezing process, it is contemplated that the section 1421 may be dissociated from substrate 1402 and stored, preserved, and/or transported in other manners as desired. As an example, the cells may be prepared for and analyzed via a variety of polymerase chain reaction (PCR) techniques to analyze genomic information such as DNA sequencing.

Chip 1400 may optionally include a plurality of chamber division lines 1422 separating the individual chambers 1414 and allowing for the dissociation of each chamber 1414 separate from the remaining chambers and the remainder of substrate 1402. In this way, each individual chamber 1414 may be stored, preserved and/or transported as desired. The division line 1420 (and optional lines 1422) may include dissociating means as is known in the art. As an example, division line 1420 (and optional lines 1422) may be perforated. As another example, division line 1420 (and optional lines 1422) may include a strip of weakened material to allow the section 1421 to be easily dissociated. In alternative embodiments, division line 1420 (and optional lines 1422) may be absent and the section 1421 may be dissociated in other appropriate manners as would occur to one of ordinary skill in the art.

As mentioned above, the chambers 1414 may contain the necessary nutrients, reagents and/or chemicals therein to maintain the cells in a healthy, viable state and/or fix the cells in their current state. In certain embodiments, the nutrients, reagents and/or chemicals in the chambers 1414 may facilitate preparation of the cells for freezing. As an example, the entire chip 1400 or just the dissociated section(s) can be placed in an automated cell cryogenic device to freeze the cells. In certain embodiments, the chip 1400 may be prepackaged with the necessary nutrients, reagents and/or chemicals in one or more of the chambers 1414.

### Microfluidic Device for Performing Functional Assays

Certain embodiments of the present disclosure are generally directed to systems for the analysis of a sample on a microfluidic device using cytometry (such as flow cytometry or image cytometry). In a process commonly known in the art as a functional assay (or kinetic assay), a reagent may need to be added to activate the sample before the cytometric analysis is executed. For example, the presence of a reagent may stimulate or cause the sample cells to change physical or chemical properties, which can then be measured to determine how the cells respond to the stimulus and therefore whether the cells are functioning properly. In certain applications, the activated sample cells may produce and excrete proteins, viruses, or other biological or chemical material. This excreted material may also be measured and analyzed to determine cell function. Unused reagent, or perhaps even reacted reagent, may also need to be washed from the sample cells either before or after the cells are analyzed.

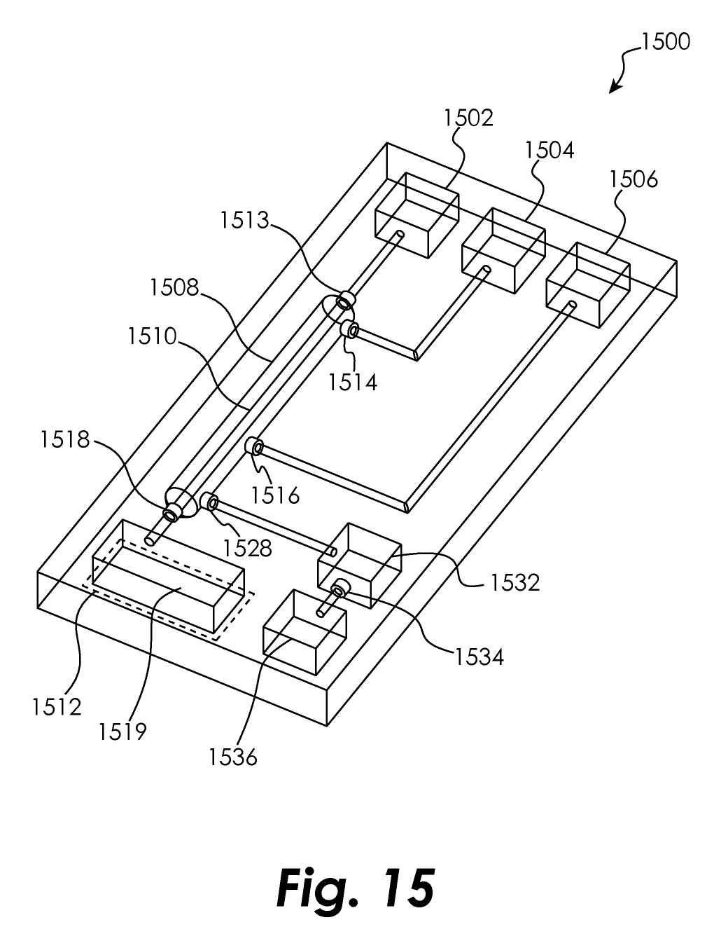

FIG. 15 illustrates a microfluidic device 1500 for analyzing samples using cytometry. The device 1500 may comprise a sample repository 1502, a reagent repository 1504, a wash solution repository 1506, a wash waste repository 1532, an outer preparation channel 1508, an inner preparation channel 1510 which is inside and preferably coaxial with outer preparation channel 1508, and a cytometry analysis section 1512. Ports 1513, 1514, 1516, 1518, 1528, and 1534 are placed as shown in FIG. 15 to allow flow between the various components. These ports contain valves that may be opened or closed by the application of appropriate control signals, as is known in the art. For simplicity and ease of illustration, FIG. 15 shows single channels extending between the components, areas or sections of device 1500. However, it should be appreciated that the single channels may be representative of multiple cytometry channels and ports and a variety of possible configurations of channels as would occur to one skilled in the art. Additionally, repositories 1502, 1504, 1506, and 1532 may instead be located external to microfluidic device 1500.

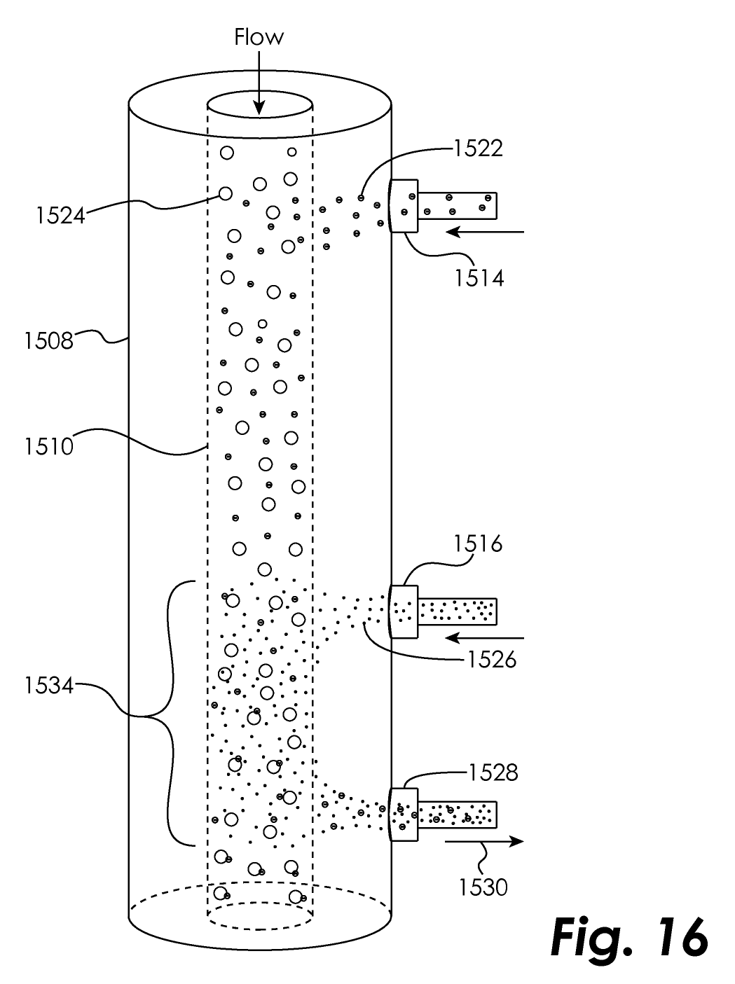

FIG. 16 shows a detail view of the outer preparation channel 1508 and the inner preparation channel 1510. In operation, port 1513 is opened to allow the sample material to flow only into the inner preparation channel 1510. The surface of the inner preparation channel 1510 may be comprised of various filtering materials known in the art which are suitable for allowing smaller molecules or cells to pass through while preventing larger ones from passing. The reagent cells or molecules are generally smaller in size than the sample cells. This allows the reagent material and wash solution to diffuse through the surface of the inner preparation channel 1510 with the sample cells 1524 remaining trapped inside the inner preparation channel 1510. After reagent material 1522 is injected into the outer preparation channel 1508 through port 1514, some of the reagent material 1522 will diffuse into the inner preparation channel 1510, causing the properties of the sample cells 1524 to change. The rate of change in such properties (e.g., color, luminescence, excretion of proteins) can then be measured to determine whether the sample cells 1510 are healthy or otherwise functioning properly. This measurement can be performed by the cytometry analysis section 1512 or by other sensors (not shown) located along the outer preparation channel 1508.

In certain applications, reagent material 1522 that has not attached itself or otherwise reacted with the sample cells 1524 will need to be washed away in a wash region 1534 before the sample cells 1524 are evaluated by the cytometry analysis section 1512. Port 1516 can be use to inject a wash solution 1526, such as phosphate buffered saline or other appropriate material, into the outer preparation channel 1508. Some portion of the wash solution 1526 will diffuse or pass through the surface of the inner preparation channel 1510. The wash solution 1526, along with any unused reagent, will be induced to pass back through the surface of the inner preparation channel 1510 near a wash extraction port 1528 and routed to the wash waste repository 1532. Various means known in the art may be used to accomplish this, such as creating a suction flow in the direction of arrow 1530. The wash waste may also be expelled from the device 1500 via an optional waste port (not shown).

In certain embodiments, the sample cells 1524 will themselves increase in size once they have reacted with the reagent 1522. If the inner preparation channel 1510 is manufactured such that the larger reacted sample cells 1524 will not pass through its surface but the unreacted sample cells 1524 will, the unreacted sample cells 1524 may also be extracted out of the inner preparation channel 1510 via the wash extraction port 1528. This ensures that only properly reacted sample cells 1524 are received by the cytometry analysis section 1512.

The activated sample cells 1524 then proceed through port 1518 into well 1519 and cytometry analysis section 1512. The cytometry analysis portion 1512 performs cytometry analysis on the received sample cells 1524. The specific operations that occur in the cytometry analysis section 1512 and the specific routing of the microfluidic channels are not critical to the present disclosure.

In certain types of assays, it is not the sample cells themselves, but rather the material excreted or produced by the activated sample cells that needs to be analyzed. For example, if the sample cells are B-cell lymphocytes, they should produce a certain antibody in the presence of a certain antigen, for example, a virus (such as mumps, or other disease virus). In that case, the virus would be the activating reagent. Viral particles are generally very small (in the range of 100 to 200 nanometers), and the B-cell lymphocytes are relatively larger (greater than two microns). A reagent containing the viral particles can then be injected into the outer preparation channel 1508 via port 1514, after which they will pass through the surface of the inner preparation channel 1510 and cause the sample cells 1524 (B-cell lymphocytes in this example) to produce certain antibodies. The wash solution 1526 will then be injected into the outer preparation channel 1508 and diffuse through the surface of the inner preparation channel 1510. The wash solution and the antibody particles will then be extracted by port 1528 by suction flow or other means as described hereinabove, with the sample cells 1524 remaining trapped within the inner preparation channel 1510.

Once extracted, port 1534 can be opened to route the antibody particles to an analysis section 1536 suitable for measuring the concentration and amount of antibody in the solution. Various methods for measuring the bulk amount of antibodies in a solution are known in the art. The antibody particles can then be analyzed to determine whether an expected quantity of antibodies was produced in relation to the amount of injected viral particles. This information is then used to determine whether a patient's B-cell lymphocytes are functioning properly, and more particularly, whether they are immune (or susceptible) to certain viruses. In certain embodiments, the produced antibodies may be harvested for use in vaccines or other medical products. Those sample cells that produce a higher amount of antibodies can be isolated using cell sorting techniques and later cloned based on their DNA analysis to produce more effective vaccines. It shall be understood that this method may be used to perform assays or harvest antibodies on other types of chemical and biological particles in addition to the ones described hereinabove. This method also allows the capture and measurement of very small quantities of antibody.

In other embodiments, the wash solution may be analyzed by cytometry section 1512. This can easily be accomplished by adding appropriate additional valves and channels (not shown) to route the material extracted by wash extraction port 1528 to the cytometry analysis section 1512.

With all of the embodiments disclosed herein, the use of a microfluidic device on a substrate offers many advantages, one of which is that the microfluidic device may be treated as a disposable part, allowing a new microfluidic device to be used for sorting each new sample of cells. This greatly simplifies the handling of the sorting equipment and reduces the complexity of cleaning the equipment to prevent cross contamination between sorting sessions, because much of the hardware through which the samples flow is simply disposed of. The microfluidic device also lends itself well to sterilization (such as by gamma irradiation) before being disposed of.

While the disclosure has been illustrated and described in detail in the drawings and foregoing description, the same is to be considered as illustrative and not restrictive in character, it being understood that only the preferred embodiments have been shown and described and that all changes and modifications that come within the spirit of the disclosure are desired to be protected.

What is claimed:

1. A microfluidic device, comprising:
a substrate;
a microfluidic flow channel formed in said substrate, wherein said flow channel extends through a portion of said substrate adapted to facilitate cytometry analysis of cells flowing in said flow channel; and
a data storage medium onboard said substrate, said data storage medium operative to store data relating to use of said microfluidic device.

2. The microfluidic device of claim 1, further comprising:
an input port fluidically coupled to said flow channel.

3. The microfluidic device of claim 1, further comprising:
a first sample well fluidically coupled to said flow channel;
a second sample well fluidically coupled to said flow channel; and
a flow diverter having a flow diverter input coupled to said flow channel, a first flow diverter outlet coupled to said first sample well, and a second flow diverter outlet coupled to said second sample well, said flow diverter having a first position and a second position;
wherein said flow diverter is operative to cause fluid in said flow channel to flow to said first sample well when said flow diverter is in a first position; and
wherein said flow diverter is operative to cause fluid in said flow channel to flow to said second sample well when said flow diverter is in a second position.

4. The microfluidic device of claim 3, wherein said flow diverter is selected from the group consisting of: piezoelectric devices, air bubble insertion means, and magnetically actuated fluid deflectors.

5. The microfluidic device of claim 1, wherein a location of said data storage medium is selected from the group consisting of: on said substrate and in said substrate.

6. The microfluidic device of claim 1, wherein said data storage medium is selected from the group consisting of: a hologram, a nonvolatile random access memory, a writeable DVD element, and a magnetic stripe.

7. The microfluidic device of claim 1, wherein said data storage medium contains information selected from the group consisting of: origin of the cells, operations performed on the cells, operations to be performed on the cells, a medical history of a patient, a pathologist report, dates a sheath fluid was manufactured, dates the cells were processed, identification of a technician performing tests, and results from processing of the cells.

8. A method of detecting cells in a sample, the method comprising the steps of:
a) providing a microfluidic device, said microfluidic device comprising:
a substrate;
a microfluidic flow channel formed in said substrate, wherein said flow channel extends through a portion of said substrate adapted to facilitate cytometry analysis of cells flowing in said flow channel; and
a data storage medium onboard said substrate, said data storage medium operative to store data relating to use of said microfluidic device;
b) performing a cytometry analysis of cells flowing in said flow channel; and
c) recording data on said data storage medium.

9. The method of claim 8, wherein said data is selected from the group consisting of: origin of the cells, operations performed on the cells, operations to be performed on the cells, a medical history of a patient, a pathologist report, dates a sheath fluid was manufactured, dates the cells were processed, identification of a technician performing tests, and results from processing of the cells.

10. A microfluidic device, comprising:
a substrate;
a microfluidic flow channel formed in said substrate, wherein said flow channel extends through a portion of said substrate adapted to facilitate cytometry analysis of cells flowing in said flow channel; and
a sample well fluidically coupled to said flow channel; and
an anticoagulant disposed in said sample well prior to introduction of a sample into said sample well.

11. A method of detecting cells in a sample, the method comprising the steps of:
a) providing a microfluidic device, said microfluidic device comprising:
a substrate;
a first microfluidic flow channel formed in said substrate, wherein said first flow channel extends through a first portion of said substrate adapted to facilitate cytometry analysis of cells flowing in said first flow channel; and
a second microfluidic flow channel formed in said substrate, wherein said second flow channel extends through a second portion of said substrate adapted to facilitate cytometry analysis of cells flowing in said second flow channel;
b) placing a test sample in said first flow channel;
c) performing a cytometry analysis of cells flowing in said first flow channel;
d) placing a control sample in said second flow channel; and
e) performing a cytometry analysis of cells flowing in said second flow channel.

12. A method of detecting cells in a sample, the method comprising the steps of:
a) providing a microfluidic device, said microfluidic device comprising:
a substrate;
a microfluidic flow channel formed in said substrate, wherein said flow channel extends through a portion of said substrate adapted to facilitate cytometry analysis of cells flowing in said flow channel;
a first well fluidically coupled to said microfluidic flow channel, said first well containing a material at a first concentration; and
a second well fluidically coupled to said microfluidic flow channel, said second well containing said material at a second concentration;
b) placing a test sample in said flow channel;
c) performing a cytometry analysis of cells flowing in said flow channel;
d) causing a first portion of said cells to enter said first well;
e) causing a second portion of said cells to enter said second well;
f) measuring a response of said first portion of said cells to said first concentration; and
g) measuring a response of said second portion of said cells to said second concentration.

13. A method of detecting cells in a sample, the method comprising the steps of:
a) providing a microfluidic device, said microfluidic device comprising:
a substrate;
a first microfluidic flow channel formed in said substrate, wherein said first flow channel extends through a first portion of said substrate adapted to facilitate cytometry analysis of cells flowing in said first flow channel; and
a second microfluidic flow channel formed in said substrate, wherein said second flow channel extends through a second portion of said substrate adapted to facilitate cytometry analysis of cells flowing in said second flow channel;
b) placing a first portion of a test sample in said first flow channel;
c) performing a cytometry analysis of cells flowing in said first flow channel;
d) placing a second portion of the test sample in said second flow channel; and
e) performing a cytometry analysis of cells flowing in said second flow channel.

14. A microfluidic device, comprising:
a substrate having a first thermal conductivity;
a microfluidic flow channel formed in said substrate, wherein said flow channel extends through a portion of said substrate adapted to facilitate cytometry analysis of cells flowing in said flow channel; and
a pad formed onboard said substrate, said pad having a second thermal conductivity;
wherein said first thermal conductivity is different than said second thermal conductivity.

15. A microfluidic device, comprising:
a substrate;
a microfluidic flow channel formed in said substrate, wherein said flow channel extends through a portion of said substrate adapted to facilitate cytometry analysis of cells flowing in said flow channel; and
an anatomy simulating region disposed within said flow channel.

16. A microfluidic device, comprising:
a substrate;
a microfluidic flow channel formed in said substrate, wherein said flow channel extends through a portion of said substrate adapted to facilitate cytometry analysis of cells flowing in said flow channel; and
a sample receiving well formed onboard said substrate and fluidically coupled to said flow channel, said sample well operative to receive a sample; and
a sample preparation well formed onboard said substrate and fluidically coupled to said flow channel, said sample preparation well containing material operative to prepare said sample for cytometry analysis.

17. The microfluidic device of claim 16, wherein said material is placed in said sample preparation well prior to said sample being placed in said sample receiving well.

18. The microfluidic device of claim 16, further comprising:
a first sorting well fluidically coupled to said flow channel;
a second sorting well fluidically coupled to said flow channel; and
a flow diverter having a flow diverter input coupled to said flow channel, a first flow diverter outlet coupled to said first sorting well, and a second flow diverter outlet coupled to said second sorting well, said flow diverter having a first position and a second position;
wherein said flow diverter is operative to cause fluid in said flow channel to flow to said first sorting well when said flow diverter is in a first position; and
wherein said flow diverter is operative to cause fluid in said flow channel to flow to said second sorting well when said flow diverter is in a second position.

19. The microfluidic device of claim 18, wherein said flow diverter is selected from the group consisting of: piezoelectric devices, air bubble insertion means, and magnetically actuated fluid deflectors.

20. The microfluidic device of claim 16, wherein a location of said sample receiving well and said sample preparation well is selected from the group consisting of: on said substrate and in said substrate.

21. The microfluidic device of claim 16, wherein said medium is selected from the group consisting of: chemicals and reagents.

22. The microfluidic device of claim 16, wherein said medium is lyophilized prior to being placed in said sample preparation well.

23. A method of analyzing cells in a sample, the method comprising the steps of:
a) providing a microfluidic device, said microfluidic device comprising:
a substrate;
a microfluidic flow channel formed in said substrate, wherein said flow channel extends through a portion of said substrate adapted to facilitate cytometry analysis of cells flowing in said flow channel; and
a sample receiving well formed onboard said substrate and fluidically coupled to said flow channel, said sample well operative to receive a sample; and
a sample preparation well formed onboard said substrate and fluidically coupled to said flow channel, said sample preparation well containing material operative to prepare said sample for cytometry analysis;
b) placing a sample in the sample receiving well;
c) causing said sample to flow in said flow channel to said sample preparation well where said sample will react with said material;
d) causing said sample to flow out of said sample preparation well and into said flow channel; and
e) performing a cytometry analysis of sample flowing in said flow channel.

24. A method of analyzing cells in a sample, the method comprising the steps of:
a) providing a microfluidic device, said microfluidic device comprising:
a substrate;
a microfluidic flow channel formed in said substrate, wherein said flow channel extends through a portion of said substrate adapted to facilitate cytometry analysis of cells flowing in said flow channel; and
a sample receiving well formed onboard said substrate and fluidically coupled to said flow channel, said sample well operative to receive a sample;
b) placing a sample in the sample receiving well; and
c) dissociating said sample receiving well from said substrate.

25. A microfluidic device, comprising:
a substrate;
a microfluidic flow channel formed in said substrate, wherein said flow channel extends through a portion of said substrate adapted to facilitate cytometry analysis of cells flowing in said flow channel;
an inner preparation channel onboard said substrate, said inner preparation channel being fluidically coupled to said flow channel; and
an outer preparation channel onboard said substrate, said outer preparation channel enclosing at least a portion of said inner preparation channel.
